(19) United States
(12) Patent Application Publication
(10) Pub. No.: US 2022/0205020 A1
(43) Pub. Date: Jun. 30, 2022

(54) COMPOSITIONS AND METHODS FOR DETECTION OF BACTERIA AND FUNGI ASSOCIATED WITH BACTERIAL AND CANDIDA VAGINOSIS

(71) Applicant: Roche Molecular Systems, Inc., Pleasanton, CA (US)

(72) Inventors: Ellen H. Fiss Hobart, Albany, CA (US); Claudia Litterst, Walnut Creek, CA (US); Sharon Ho-Chen Chiu, Pleasanton, CA (US); Nancy Patten, Emeryville, CA (US); Ha Bich Tran, Daly City, CA (US); Julie Tsai, Newark, CA (US); Rui Zhang, Dublin, CA (US)

(21) Appl. No.: 17/560,724

(22) Filed: Dec. 23, 2021

Related U.S. Application Data
(60) Provisional application No. 63/132,438, filed on Dec. 30, 2020.
(60) Provisional application No. 63/263,717, filed on Nov. 8, 2021.

(51) Int. Cl. C12Q 1/689

(52) U.S. Cl. CPC C12Q 1/689

(57) ABSTRACT

Methods for the rapid detection of the presence or absence of a plurality of Bacterial Vaginosis-related (BV-related) bacteria and/or vulvovaginal candidiasis (VVC)-associated Candida in a biological or non-biological sample are described. The methods can include performing an amplifying step, a hybridizing step, and a detecting step. Furthermore, primers and probes targeting specific genes and kits are provided that are designed for the detection of BV-related bacteria and VVC-associated Candida.

### CROSS REFERENCE TO RELATED APPLICATIONS

This application claims the benefit of priority to U.S. Provisional Application No. 63/132,438, filed on Dec. 30, 2020, and U.S. Provisional Application No. 63/263,717, filed on Nov. 8, 2021, each of which is hereby incorporated in its entirety by reference.

### REFERENCE TO SEQUENCE LISTING

This application contains a Sequence Listing submitted as an electronic text file named “36606_US2_ST25.txt”, having a size in bytes of 32 kb, and created on Dec. 1, 2021.

### FIELD OF THE INVENTION

The present disclosure relates to the field of molecular diagnostics, and more particularly to detection of various bacterial and fungal strains that are associated with bacterial and Candida vaginosis.

### BACKGROUND OF THE INVENTION

Vaginitis is responsible for as many as 50% of all gynecologic visits in the United States and represents a major contributor to health care expenses. Infectious vaginitis due to bacterial vaginosis (BV), vulvovaginal candidiasis (VVC) and trichomoniasis accounts for up to 90% of these cases (2). Unlike trichommoniasis, both BV and VVC are attributable to several pathogens. For VVC, overgrowth of Candida albicans is predominant, although other Candida species, including Candida glabrata, may contribute as well. BV is harder to diagnose because the pathogenesis involves decreased levels of Lactobacillus bacteria concomitant with increased concentrations of BV-associated bacteria, such as Gardnerella vaginalis, Mobiluncus spp., and Atopobium vaginae.

Various diagnostic methods are available to identify the underlying cause of vaginitis.

In the clinician's office, a combination of pH a potassium hydroxide (KOH) test, and microscopic examination of fresh samples of vaginal discharge are routinely used, despite their relatively poor performance. For BV, diagnosis often relies on the use of either clinical Amsel criteria or Gram stain and Nugent score (considered the gold standard laboratory method for diagnosis of BV). Examination of wet mounts with KOH preparation and/or vaginal cultures for Candida are the most common diagnostic tools for VVC. Highly sensitive and specific nucleic acid amplification tests (NAATs) are recommended for detecting Trichomonas vaginalis, but examination of wet-mount preparations is still commonly used in clinical practice. However, several barriers are associated with the use of nonmolecular methods, including lack of equipment in the clinic, subjectivity of the clinical endpoints used and inconsistent employment between practitioners, lack of proper training in microscopy, and overall poor sensitivity of the tests. Diagnosis of the underlying cause of vaginitis is further complicated by the common symptomatology reported for BV, VVC, and trichomoniasis the incidence of mixed infections or coinfections; and the recurrence of vaginal symptoms.

Accordingly, there is a need for developing more efficient and faster methods for detecting vulvovaginal candidiasis and bacterial vaginosis, for example a method allowing detecting of both vaginal disorders in a single assay, in order to effectively deliver proper treatments to patients.

### SUMMARY OF THE INVENTION

In one aspect, a method to detect a plurality of BV-related bacteria in a biological sample is disclosed, wherein the plurality of BV-related bacteria comprises Lactobacillus spp., Gardnerella vaginalis, and at least one from Atopobium vaginae, Megasphaera Type 1, Eggerthella spp., Prevotella spp. and Bacterial Vaginosis Associated Bacterium BVAB-2.

In some embodiments, the method comprises performing an amplifying step comprising contacting the sample with a set of primers to produce an amplification product if a nucleic acid from the BV-related bacteria is present in the sample; performing a hybridizing step comprising contacting each amplification product with one or more detectable probes; and detecting the presence of each amplification product, wherein the presence of the amplification product is indicative of the presence of the BV-related bacteria in the sample; wherein the set of primers to produce an amplification product from Lactobacillus spp. comprises a forward primer selected from SEQ ID NOs: 38, 41, or 44, and a reverse primer selected from SEQ ID NOs: 39, 42 or 45, 47 or 73, and the one or more detectable probes is selected from SEQ ID NOs: 40, 43, 46 and 74; wherein the set of primers to produce an amplification product from Gardnerella vaginalis comprises a forward primer selected from SEQ ID NOs: 1, 4, 5, 8 or 11, and a reverse primer selected from SEQ ID NOs: 2, 6, 9, or 12, and the one or more detectable probes is selected from SEQ ID NOs: 3, 7, 10 and 13; wherein the set of primers to produce an amplification product from Atopobium vaginae comprises a forward primer selected from SEQ ID NOs: 14, 17, 20, 67 or 70, and a reverse primer selected from SEQ ID NOs: 15, 18, 21, 68 or 71, and the one or more detectable probes is selected from SEQ ID NOs: 16, 19, 22, 66, 69 and 72; wherein the set of primers to produce an amplification product from Megasphaera Type 1 comprises a forward primer selected from SEQ ID NOs: 23, 26 or 27, and a reverse primer selected from SEQ ID NOs: 24 or 28, and the one or more detectable probe is selected from SEQ ID NOs: 25 and 29; wherein the set of primers to produce an amplification product from Eggerthella spp. comprises a forward primer selected from SEQ ID NOs: 84 or 87, and a reverse primer selected from SEQ ID NOs: 85 or 88, and the one or more detectable probe is selected from SEQ ID NOs: 86 and 89; wherein the set of primers to produce an amplification product from Prevotella spp. comprises a forward primer selected from SEQ ID NOs: 90, 95, 96 or 97, and a reverse primer selected from SEQ ID NOs: 91, 92, 93, 98, 99 or 100, and the one or more detectable probe is selected from SEQ ID NOs: 94, 101 and 102; and wherein the set of primers to produce an amplification product from BVAB-2 comprises a forward primer selected from SEQ ID NOs: 30 or 33, and a reverse primer selected from SEQ ID NOs: 31, 34 or 36, and the one or more detectable probe is selected from SEQ ID NOs: 32, 35 and 37.

In some embodiments, the plurality of BV-related bacteria are Lactobacillus spp., Gardnerella vaginalis, and Atopobium vaginae, wherein the set of primers to produce an amplification product from Lactobacillus spp. comprises a forward primer selected from SEQ ID NOs: 38, 41, or 44, and a reverse primer selected from SEQ ID NOs: 39, 42 or 45, 47 or 73, and the one or more detectable probes is selected from SEQ ID NOs: 40, 43, 46 and 74; wherein the set of primers to produce an amplification product from Gardnerella vaginalis comprises a forward primer selected from SEQ ID NOs: 1, 4, 5, 8 or 11, and a reverse primer selected from SEQ ID NOs: 2, 6, 9, or 12, and the one or more detectable probes is selected from SEQ ID NOs: 3, 7, 10 and 13; and wherein the set of primers to produce an amplification product from Atopobium vaginae comprises a forward primer selected from SEQ ID NOs: 14, 17, 20, 67 or 70, and a reverse primer selected from SEQ ID NOs: 15, 18, 21, 68 or 71, and the one or more detectable probes is selected from SEQ ID NOs: 16, 19, 22, 66, 69 and 72.

In some embodiments, the set of primers to produce an amplification product from Lactobacillus spp. comprises a forward primer of SEQ ID NO: 44, and a reverse primer of SEQ ID NO: 45 and the one or more detectable probes is SEQ ID NO: 46; and the set of primers to produce an amplification product from Gardnerella vaginalis comprises a forward primer of SEQ ID NO: 11, and a reverse primer of SEQ ID NO: 12, and the one or more detectable probes is SEQ ID NO: 13; and the set of primers to produce an amplification product from Atopobium vaginae comprises a forward primer of SEQ ID NO: 20, and a reverse primer of SEQ ID NO: 21, and the one or more detectable probes is SEQ ID NO: 72.

In other embodiments, the hybridizing step of the present methods comprises contacting the amplification product with the detectable probe that is labeled with a donor fluorescent moiety and a corresponding acceptor moiety; and the detecting step comprises detecting the presence or absence of fluorescence resonance energy transfer (FRET) between the donor fluorescent moiety and the acceptor moiety of the probe, wherein the presence or absence of fluorescence is indicative of the presence or absence of the BV-related bacteria in the sample. In a further embodiment, the amplification step of the present methods employs a polymerase enzyme having 5′ to 3′ nuclease activity.

In some embodiments, the “contacting” step further comprises contacting said biological sample and said primers with DNA polymerase, a plurality of free nucleotides comprising adenine, thymine, cytosine and guanine, and/or a buffer to produce a reaction mixture. The nucleic acids extracted from the biological sample may comprise or consist of double stranded DNA. A reaction mixture may optionally further contain bivalent cations, monovalent cation potassium ions, one or more detectably labeled probes, and/or any combination thereof. In some embodiments, the “generating amplicons” step involves (a) heating the reaction mixture to a first predetermined temperature for a first predetermined period of time to separate strands of double stranded DNA present in the biological sample or in the nucleic acids, (b) cooling the reaction mixture to a second predetermined temperature for a second predetermined time under conditions to allow the primers to hybridize with their complementary sequences and to allow the DNA polymerase to extend the primers, and (c) repeating steps (a) and (b) at least 10 to 12 times. In some embodiments, steps (a) and (b) are repeated at least 15, 20, 22 or 25 times.

In some embodiments, the sample is a biological sample. In some embodiments, the biological sample is collected from the urethra, penis, anus, throat, cervix, or vagina. In some embodiments, the biological sample is DNA, RNA or total nucleic acids extracted from a clinical specimen.

In some embodiments the hybridizing step comprises contacting the amplification product with the detectable gene probe that is labeled with a donor fluorescent moiety and a corresponding acceptor moiety; and the detecting step comprises detecting the presence or absence of fluorescence resonance energy transfer (FRET) between the donor fluorescent moiety and the acceptor moiety of the probe, wherein the presence or absence of fluorescence is indicative of the presence or absence of BV-related bacteria in the sample. In some embodiments the amplifying and the hybridizing steps are repeated. Herein, the number of repetitions depends, e.g., on the nature of the sample. If the sample is a complex mixture of nucleic acids, more amplifying and hybridizing steps will be required to amplify the target sequence sufficient for detection. In some embodiments, the amplifying and the hybridizing steps are repeated at least about 20 times, but may be repeated as many as at least 25, 30, 40, 50, 60, or even 100 times. Further, detecting the presence or absence of the amplification product may be performed during or after each amplifying and hybridizing step, during or after every other amplifying and hybridizing step, during or after particular amplifying and hybridizing steps or during or after particular amplifying and hybridizing steps, in which—if present—sufficient amplification product for detection is expected. In some embodiments, the amplifying step employs a polymerase enzyme having 5′ to 3′ nuclease activity. In some embodiments, the donor fluorescent moiety and the corresponding acceptor moiety are within no more than 8-20 nucleotides of each other on the probe. In some embodiments, the acceptor moiety is a quencher. In some embodiments the oligonucleotides comprise or consist of a sequence of nucleotides selected from SEQ ID NOs: 1-47, 66-74, and 84-102 or a complement thereof have 100 or fewer nucleotides, 50 or fewer nucleotides, 40 or fewer nucleotides or 30 or fewer nucleotides.

The present disclosure also provides a kit for detecting Bacterial Vaginosis-related (BV-related) bacteria in a sample comprising: a forward primer comprising a first oligonucleotide sequence selected from the group consisting of SEQ ID NOs: 1, 4, 5, 8, 11, 14, 17, 20, 23, 26, 27, 30, 33, 38, 41, 44, 67, 70, 84, 87, 90, 95, 96, and 97; a reverse primer comprising a second oligonucleotide sequence selected from the group consisting of SEQ ID NOs: 2, 6, 9, 12, 15, 18, 21, 24, 28, 31, 34, 36, 39, 42, 45, 47, 68, 71, 73, 85, 88, 91, 92, 93, 98, 99, and 100; and a third detectably labeled oligonucleotide sequence selected from the group consisting of SEQ ID NOs: 3, 7, 10, 13, 16, 66, 69, 19, 22, 72, 25, 29, 32, 35, 37, 40, 43, 46, 74, 86, 89, 94, 101, and 102, or a complement thereof, the third detectably labeled oligonucleotide sequence configured to hybridize to an amplicon generated by the forward primer and the reverse primer. In some embodiments, the third detectably labeled oligonucleotide sequence comprises a donor fluorescent moiety and a corresponding acceptor moiety. In some embodiments, the kit further comprises nucleoside triphosphates, nucleic acid polymerase, and buffers necessary for the function of the nucleic acid polymerase. In yet other embodiments, at least one of the first, second, and third oligonucleotide sequences comprises at least one modified nucleotide.

In one embodiment, the BV-related bacteria, comprises Lactobacillus spp., Gardnerella vaginalis, Atopobium vaginae, Megasphaera Type 1, Eggerthella spp., Prevotella spp. and BVAB-2. In some embodiments, the kit comprises primers and probes capable of hybridizing to the 23s rRNA or to the D-LDH genes of Lactobacillus spp., that comprises a sequence selected from SEQ ID NOs: 38-47 and 73-74. In some embodiments, the kit comprises primers and probes capable of hybridizing to the tuf gene, or the 23s rRNA gene or the 16s rRNA gene of Gardnerella vaginalis, that comprise a sequence selected from SEQ ID NOs: 1-13. In some embodiments, the kit comprises primers and probes capable of hybridizing to the 23s rRNA gene or the tufA gene of Atopobium vaginae, that comprise a sequence selected from SEQ ID NOs: 14-22 and 66-72. In some embodiments, the kit comprises primers and probes capable of hybridizing to the 16s rRNA gene of Megasphaera type 1, that comprise a sequence selected from SEQ ID NOs: 23-29. In some embodiments, the kit comprises primers and probes capable of hybridizing to the hybridizing to the 16s rRNA gene of Eggerthella spp., that comprise a sequence selected from SEQ ID NOs: 84-89. In some embodiments, the kit comprise primers and probes capable of hybridizing to the 16s rRNA gene of Prevotella spp., that comprise a sequence selected from SEQ ID NOs: 90-102. In some embodiments, the kit comprises primers and probes capable of hybridizing to the 16s rRNA gene of BVAB-2, that comprise a sequence selected from SEQ ID NOs: 30-37.

In another aspect, the present disclosure provides a method to detect vulvovaginal candidiasis (VVC)-associated Candida species in a biological sample, wherein the VVC-associated Candida species comprises Candida albicans, Candida tropicalis, Candida dubliniensis, and Candida parapsilosis, (collectively referred as Candida spp.). In some embodiments, the VVC-associated species further comprises Candida krusei and/or Candida glabrata.

In some embodiments, the method comprises performing an amplifying step comprising contacting the sample with a set of primers to produce an amplification product if a nucleic acid from the VVC-associated Candida is present in the sample; performing a hybridizing step comprising contacting each amplification product with one or more detectable probes; and detecting the presence of each amplification product, wherein the presence of the amplification product is indicative of the presence of the VVC-associated Candida in the sample; wherein the set of primers to produce an amplification product from Candida spp. comprises a forward primer selected from SEQ ID NOs: 48, 51, 54, 57 or 76, and a reverse primer selected from SEQ ID NOs: 49, 52, 55, 58 or 77, and the one or more detectable probes is selected from SEQ ID NOs: 50, 53, 56, 59, 75 and 78; wherein the set of primers to produce an amplification product from Candida krusei comprises a forward primer selected from SEQ ID NOs: 60, 79 or 82, and a reverse primer selected from SEQ ID NOs: 61, 80 or 83, and the one or more detectable probes is selected from SEQ ID NOs: 62 and 81; and wherein the set of primers to produce an amplification product from Candida glabrata comprises a forward primer of SEQ ID NO: 63, and a reverse primer of SEQ ID NO: 64, and the one or more detectable probes is SEQ ID NO; 65.

In some embodiments, the set of primers to produce an amplification product from Candida spp. comprises a forward primer of SEQ ID NO: 76, and a reverse primer of SEQ ID NO: 77 and the one or more detectable probes is SEQ ID NO: 78; and the set of primers to produce an amplification product from Candida krusei comprises a forward primer of SEQ ID NO: 79, and a reverse primer of SEQ ID NO: 80, and the one or more detectable probes is SEQ ID NO: 81.

In other embodiments, the hybridizing step comprises contacting the amplification product with the detectable probe that is labeled with a donor fluorescent moiety and a corresponding acceptor moiety; and the detecting step comprises detecting the presence or absence of fluorescence resonance energy transfer (FRET) between the donor fluorescent moiety and the acceptor moiety of the probe, wherein the presence or absence of fluorescence is indicative of the presence or absence of the VVC-associated Candida in the sample. In a further embodiment, the amplification step employs a polymerase enzyme having 5′ to 3′ nuclease activity.

In some embodiments, the “contacting” step further comprises contacting said biological sample and said primers with DNA polymerase, a plurality of free nucleotides comprising adenine, thymine, cytosine and guanine, and/or a buffer to produce a reaction mixture. The nucleic acids extracted from the biological sample may comprise or consist of double stranded DNA. A reaction mixture may optionally further contain bivalent cations, monovalent cation potassium ions, one or more detectably labeled probes, and/or any combination thereof. In some embodiments, the “generating amplicons” step involves (a) heating the reaction mixture to a first predetermined temperature for a first predetermined period of time to separate strands of double stranded DNA present in the biological sample or in the nucleic acids, (b) cooling the reaction mixture to a second predetermined temperature for a second predetermined time under conditions to allow the primers to hybridize with their complementary sequences and to allow the DNA polymerase to extend the primers, and (c) repeating steps (a) and (b) at least 10 to 12 times. In some embodiments, steps (a) and (b) are repeated at least 15, 20, 22 or 25 times.

The present disclosure also provides a kit for detecting vulvovaginal candidiasis (VVC)-associated Candida species in a sample comprising a forward primer comprising a first oligonucleotide sequence selected from the group consisting of SEQ ID NOs: 48, 51, 54, 57, 76, 60, 79, 82, and 63; a reverse primer comprising a second oligonucleotide sequence selected from the group consisting of SEQ ID NOs: 49, 52, 55, 58, 77, 61, 80, 83, and 64; and a third detectably labeled oligonucleotide sequence selected from the group consisting of SEQ ID NOs: 50, 53, 75, 56, 59, 78, 62, 81, and 65, or a complement thereof, the third detectably labeled oligonucleotide sequence configured to hybridize to an amplicon generated by the forward primer and the reverse primer.

In some embodiments, the third detectably labeled oligonucleotide sequence comprises a donor fluorescent moiety and a corresponding acceptor moiety. In some embodiments, the kit further comprises nucleoside triphosphates, nucleic acid polymerase, and buffers necessary for the function of the nucleic acid polymerase. In yet other embodiments, at least one of the first, second, and third oligonucleotide sequences comprises at least one modified nucleotide.

In some embodiments, the VVC-associated Candida species comprises Candida albicans, Candida tropicalis, Candida dubliniensis, and Candida parapsilosis, (collectively referred as Candida spp.), Candida krusei and Candida glabrata. In some embodiments, the kit comprises primers and probes capable of hybridizing to the ribosomal RNA (rRNA) gene or to the 25s rRNA of Candida spp. that comprise a sequence selected from SEQ ID NOs: 48-59 and 75-78. In some embodiments, the kit comprises primers and probes capable of hybridizing to the Internal transcribed spacer (ITS) of the rRNA gene of Candida krusei that comprise a sequence selected from SEQ ID NOs: 60-62 and 79-83. In some embodiments, the kit comprises primers and probes capable of hybridizing to the Internal transcribed spacer (ITS) of the rRNA gene of Candida glabrata that comprise a sequence selected from SEQ ID NOs: 63-65.

In some embodiments, the sample is a biological sample. In some embodiments, the biological sample is collected from the urethra, penis, anus, throat, cervix, or vagina. In some embodiments, the biological sample is DNA, RNA or total nucleic acids extracted from a clinical specimen.

In another embodiment, the present disclosure provides an oligonucleotide that includes a nucleic acid having at least 70% sequence identity (e.g., at least 75%, 80%, 85%, 90% or 95%, etc.) to one of SEQ ID NOs: 1-102, or a complement thereof, which oligonucleotide has 100 or fewer nucleotides. Generally, these oligonucleotides may be primer nucleic acids, probe nucleic acids, or the like in these embodiments. In some embodiments, the oligonucleotides comprise at least one modified nucleotide, e.g., to alter nucleic acid hybridization stability relative to unmodified nucleotides. In some embodiments, the at least one modified nucleotide is selected from the group consisting of a N⁶-benzyl-dA, a N⁴-benzyl-dC, a N⁶-para-tert-butyl-benzyl-dA, and a N⁴-para-tert-butyl-benzyl-dC. Optionally, the oligonucleotides comprise at least one label and/or at least one quencher moiety. In some embodiments, the oligonucleotides include at least one conservatively modified variation. “Conservatively modified variations” or, simply, “conservative variations” of a particular nucleic acid sequence refers to those nucleic acids, which encode identical or essentially identical amino acid sequences, or, where the nucleic acid does not encode an amino acid sequence, to essentially identical sequences. One of skill will recognize that individual substitutions, deletions or additions which alter, add or delete a single amino acid or a small percentage of amino acids (typically less than 5%, more typically less than 4%, 2% or 1%) in an encoded sequence are “conservatively modified variations” where the alterations result in the deletion of an amino acid, addition of an amino acid, or substitution of an amino acid with a chemically similar amino acid. In some embodiments, at least one of the first and second target gene primers and detectable target gene probe comprises at least one modified nucleotide.

In one aspect, the present disclosure provides for a method of simultaneously detecting Lactobacillus spp., Gardnerella vaginalis, Atopobium vaginae, and at least one of the group selected from Candida spp., Candida krusei and Candida glabrata in a sample, comprising performing an amplifying step comprising contacting the sample with a set of primers to produce an amplification product if a nucleic acid from Lactobacillus spp., Gardnerella vaginalis, Atopobium vaginae, and at least one of the group selected from Candida spp., Candida krusei and Candida glabrata is present in the sample; performing a hybridizing step comprising contacting each amplification product with one or more detectable probes; and detecting the presence of each amplification product, wherein the presence of the amplification product is indicative of the presence of Lactobacillus spp., Gardnerella vaginalis, Atopobium vaginae, and at least one of the group selected from Candida spp., Candida krusei and Candida glabrata in the sample; wherein the set of primers to produce an amplification product from Lactobacillus spp. comprises a forward primer of SEQ ID NO: 44, and a reverse primer of SEQ ID NO: 45 and the one or more detectable probes is SEQ ID NO: 46; and the set of primers to produce an amplification product from Gardnerella vaginalis comprises a forward primer of SEQ ID NO: 11, and a reverse primer of SEQ ID NO: 12, and the one or more detectable probes is SEQ ID NO: 13; and the set of primers to produce an amplification product from Atopobium vaginae comprises a forward primer of SEQ ID NO: 20, and a reverse primer of SEQ ID NO: 21, and the one or more detectable probes is SEQ ID NO: 72; and wherein the set of primers to produce an amplification product from Candida spp. comprises a forward primer of SEQ ID NO: 76, and a reverse primer of SEQ ID NO: 77 and the one or more detectable probes is SEQ ID NO: 78; and the set of primers to produce an amplification product from Candida krusei comprises a forward primer of SEQ ID NO: 79, and a reverse primer of SEQ ID NO: 80, and the one or more detectable probes is SEQ ID NO: 81; and the set of primers to produce an amplification product from Candida glabrata comprises a forward primer of SEQ ID NO: 63, and a reverse primer of SEQ ID NO: 64, and the one or more detectable probes is SEQ ID NO: 65.

Unless otherwise defined, all technical and scientific terms used herein have the same meaning as commonly understood by one of ordinary skill in the art to which this invention belongs. Although methods and materials similar or equivalent to those described herein can be used in the practice or testing of the present subject matter, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### BRIEF DESCRIPTION OF THE DRAWINGS

FIG. 1A shows the growth curves of the PCR experiment for amplifying and detecting Atopobium vaginae as described in Example 2. FIG. 1B shows the calculated Ct values of the same experiment.

FIG. 2 shows the calculated Ct values of the PCR experiment for amplifying and detecting Gardnerella vaginalis as described in Example 3.

FIG. 3 shows the calculated Ct values of the PCR experiment for amplifying and detecting BVAB-2 as described in Example 4.

FIG. 4 shows the calculated Ct values of the PCR experiment for amplifying and detecting Megasphaera type 1 as described in Example 5.

FIG. 5 shows the calculated Ct values of the PCR experiment for amplifying and detecting Lactobacillus spp. as described in Example 6.

FIG. 6 shows the calculated Ct values of the PCR experiment in the CAPT_rRNA_1 assay for amplifying and detecting Candida spp. as described in Example 9.

FIG. 7 shows the calculated Ct values of the PCR experiment in the CVS_rRNA_1 assay for amplifying and detecting Candida spp. and Candida glabrata as described in Example 9.

FIG. 8 shows the growth curves in Channel 1 for the detection of 16s rRNA of Gardnerella vaginalis in the multiplex PCR assay as described in Example 10. T1-4Ab: pool of plasmids with Gardnerella vaginalis 16s, Lactobacillus crispatus LDH, Atopobium vaginae tufA, Candida albicans 25s rRNA sequences, and human genomic DNA. T1-4Gb: pool of plasmids with Gardnerella vaginalis 16s, Lactobacillus crispatus LDH, Atopobium vaginae tufA, Candida glabrata ITS sequences, and human genomic DNA. HIVD: sample diluent.

FIG. 9 shows the growth curves in Channel 2 for the detection of D-LDH gene of Lactobacillus spp. in the multiplex PCR assay as described in Example 10. T1-4Ab: pool of plasmids with Gardnerella vaginalis 16s, Lactobacillus crispatus LDH, Atopobium vaginae tufA, Candida albicans 25s rRNA sequences, and human genomic DNA. T1-4Gb: pool of plasmids with Gardnerella vaginalis 16s, Lactobacillus crispatus LDH, Atopobium vaginae tufA, Candida glabrata ITS sequences, and human genomic DNA. HIVD: sample diluent.

FIG. 10 shows the growth curves in Channel 3 for the detection of the tufA gene of Atopobium vaginae in the multiplex PCR assay as described in Example 10. T1-4Ab: pool of plasmids with Gardnerella vaginalis 16s, Lactobacillus crispatus LDH, Atopobium vaginae tufA, Candida albicans 25s rRNA sequences, and human genomic DNA. T1-4Gb: pool of plasmids with Gardnerella vaginalis 16s, Lactobacillus crispatus LDH, Atopobium vaginae tufA, Candida glabrata ITS sequences, and human genomic DNA. HIVD: sample diluent.

FIG. 11 shows the growth curves in Channel 4 for the detection of 25s rRNA of Candida spp. in the multiplex PCR assay as described in Example 10. T1-4Ab: pool of plasmids with Gardnerella vaginalis 16s, Lactobacillus crispatus LDH, Atopobium vaginae tufA, Candida albicans 25s rRNA sequences, and human genomic DNA. T1-4Gb: pool of plasmids with Gardnerella vaginalis 16s, Lactobacillus crispatus LDH, Atopobium vaginae tufA, Candida glabrata ITS sequences, and human genomic DNA. HIVD: sample diluent.

### DETAILED DESCRIPTION OF THE INVENTION

Provided herein are methods and compositions for the detection of vulvovaginal candidiasis (VVC) and bacterial vaginosis (BV). For example, primers and probes that can bind to specific genes of Candida species associated with VVC, and BV-related bacteria are provided to determine the presence or absence of the VVC-associated Candida species and BV-related bacteria in a sample, such as a biological sample. In some embodiments, multiplex nucleic acid amplification can be performed to allow the detection of VVC-associated Candida species and BV-related bacteria in a single assay.

The disclosed methods may include performing at least one cycling step that includes amplifying one or more portions of the nucleic acid molecule gene target from a sample using one or more pairs of primers. “Primer(s)” as used herein refer to oligonucleotide primers that specifically anneal to the target gene in a bacterial species or a Candida species, and initiate DNA synthesis therefrom under appropriate conditions producing the respective amplification products. Each of the discussed primers anneals to a target within or adjacent to the respective target nucleic acid molecule such that at least a portion of each amplification product contains nucleic acid sequence corresponding to the target. The one or more amplification products are produced provided that one or more of the target gene nucleic acid is present in the sample, thus the presence of the one or more of target gene amplification products is indicative of the presence of bacterial or Candida Species in the sample. The amplification product should contain the nucleic acid sequences that are complementary to one or more detectable probes for the target gene. “Probe(s)” as used herein refer to oligonucleotide probes that specifically anneal to nucleic acid sequence encoding the target gene. Each cycling step includes an amplification step, a hybridization step, and a detection step, in which the sample is contacted with the one or more detectable probes for detection of the presence or absence of the bacterial species or the Candida species in the sample.

As used herein, the term “amplifying” refers to the process of synthesizing nucleic acid molecules that are complementary to one or both strands of a template nucleic acid molecule. Amplifying a nucleic acid molecule typically includes denaturing the template nucleic acid, annealing primers to the template nucleic acid at a temperature that is below the melting temperatures of the primers, and enzymatically elongating from the primers to generate an amplification product. Amplification typically requires the presence of deoxyribonucleoside triphosphates, a DNA polymerase enzyme (e.g., Platinum® Taq) and an appropriate buffer and/or co-factors for optimal activity of the polymerase enzyme (e.g., MgCl₂ and/or KCl).

The term “primer” as used herein is known to those skilled in the art and refers to oligomeric compounds, primarily to oligonucleotides but also to modified oligonucleotides that are able to “prime” DNA synthesis by a template-dependent DNA polymerase, i.e., the 3′-end of the, e.g., oligonucleotide provides a free 3′—OH group whereto further “nucleotides” may be attached by a template-dependent DNA polymerase establishing 3′ to 5′ phosphodiester linkage whereby deoxynucleoside triphosphates are used and whereby pyrophosphate is released. Therefore, there is—except possibly for the intended function—no fundamental difference between a “primer”, an “oligonucleotide”, or a “probe”.

The term “hybridizing” refers to the annealing of one or more probes to an amplification product. Hybridization conditions typically include a temperature that is below the melting temperature of the probes but that avoids non-specific hybridization of the probes.

The term “5′ to 3′ nuclease activity” refers to an activity of a nucleic acid polymerase, typically associated with the nucleic acid strand synthesis, whereby nucleotides are removed from the 5′ end of nucleic acid strand.

The term “thermostable polymerase” refers to a polymerase enzyme that is heat stable, i.e., the enzyme catalyzes the formation of primer extension products complementary to a template and does not irreversibly denature when subjected to the elevated temperatures for the time necessary to effect denaturation of double-stranded template nucleic acids. Generally, the synthesis is initiated at the 3′ end of each primer and proceeds in the 5′ to 3′ direction along the template strand. Thermostable polymerases have been isolated from Thermus flavus, T. ruber, T. thermophilus, T. aquaticus, T. lacteus, T. rubens, Bacillus stearothermophilus, and Methanothermus fervidus. Nonetheless, polymerases that are not thermostable also can be employed in PCR assays provided the enzyme is replenished.

The term “complement thereof” refers to nucleic acid that is both the same length as, and exactly complementary to, a given nucleic acid.

The term “extension” or “elongation” when used with respect to nucleic acids refers to when additional nucleotides (or other analogous molecules) are incorporated into the nucleic acids. For example, a nucleic acid is optionally extended by a nucleotide incorporating biocatalyst, such as a polymerase that typically adds nucleotides at the 3′ terminal end of a nucleic acid.

The terms “identical” or percent “identity” in the context of two or more nucleic acid sequences, refer to two or more sequences or subsequences that are the same or have a specified percentage of nucleotides that are the same, when compared and aligned for maximum correspondence, e.g., as measured using one of the sequence comparison algorithms available to persons of skill or by visual inspection. Exemplary algorithms that are suitable for determining percent sequence identity and sequence similarity are the BLAST programs, which are described in, e.g., Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:131-141, Altschul et al. (1997) “Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656, which are each incorporated herein by reference.

A “modified nucleotide” in the context of an oligonucleotide refers to an alteration in which at least one nucleotide of the oligonucleotide sequence is replaced by a different nucleotide that provides a desired property to the oligonucleotide. Exemplary modified nucleotides that can be substituted in the oligonucleotides described herein include, e.g., a C5-methyl-dC, a C5-ethyl-dC, a C5-methyl-dU, a C5-ethyl-dU, a 2,6-diaminopurine, a C5-propynyl-dC, a C5-propynyl-dU, a C7-propynyl-dA, a C7-propynyl-dG, a C5-propargylamino-dC, a C5-propargylamino-dU, a C7-propargylamino-dA, a C7-propargylamino-dG, a 7-deaza-2-deoxyxanthosine, a pyrazolopyrimidine analog, a pseudo-dU, a nitro pyrrole, a nitro indole, 2′-O-methyl Ribo-U, 2′-O-methyl Ribo-C, an N4-ethyl-dC, an N⁶-methyl-dA, a N⁶-benzyl-dA, a N⁴-benzyl-dC, a N⁶-para-tert-butyl-benzyl-dA, a N⁴-para-tert-butyl-benzyl-dC, and the like. Many other modified nucleotides that can be substituted in the oligonucleotides are referred to herein or are otherwise known in the art. In certain embodiments, modified nucleotide substitutions modify melting temperatures (Tm) of the oligonucleotides relative to the melting temperatures of corresponding unmodified oligonucleotides. To further illustrate, certain modified nucleotide substitutions can reduce non-specific nucleic acid amplification (e.g., minimize primer dimer formation or the like), increase the yield of an intended target amplicon, and/or the like in some embodiments. Examples of these types of nucleic acid modifications are described in, e.g., U.S. Pat. No. 6,001,611, which is incorporated herein by reference.

### Detection of Bacteria and Fungi

As described herein, nucleic acid amplifications can be performed to determine the presence, absence and/or level of Candida species, and/or BV-related bacteria in a sample. Some Candida species are known to be associated with VVC, including but not limited to C. albicans, C. tropicalis, C. dubliniensis, C. parapsilosis, C. krusei, and C. glabrata. Many bacteria are also known to be related to BV, including but not limited to, Lactobacillus spp. (for example Lactobacillus crispatus (L. crispatus), Lactobacillus jensenii (L. jensenii), and Lactobacillus gasseri (L. gasseri)), Gardnerella vaginalis (G. vaginalis), Atopobium vaginae, Megasphaera Type 1 (Megasphaera-1), and BVAB-2. In some embodiments, the presence, absence and/or level of VVC-associated Candida species and BV-related bacteria is determined by detecting one or more target genes of each of the target organisms using methods known in the art, such as DNA amplifications. In some embodiments, a multiplex PCR can be performed to detect the presence, absence or level for each of the target Candida species, and/or BV-related bacteria. In some embodiments, a multiplex PCR is performed to detect the presence, absence and/or level for each of target VVC-associated Candida species, L. crispatus, L. jensenii, L. gasseri, G. vaginalis, Atopobium vaginae, Megasphaera Type 1, and BVAB-2. In some embodiments, the VVC-associated Candida species are C. albicans, C. tropicalis, C. dubliniensis, C. parapsilosis, C. krusei, and C. glabrata.

In another embodiment, nucleic acid amplifications can be performed in the same sample to determine the presence, absence and/or level of Trichomonas vaginalis (TV). Compositions and methods for the rapid detection of the presence or absence of Trichomonas vaginalis in a biological or non-biological sample are described in U.S. Patent Application Publication Number 2017/0342508, which is incorporated herein by reference in its entirety.

Each of the target VVC-associated Candida species and BV-related bacteria can be detected using separate channels in DNA amplifications. In some cases, it can be desirable to use a single fluorescence channel for detecting the presence, absence, and/or level of two or more of the VVC-associated Candida species and BV-related bacteria. For example, a single fluorescence channel can be used to detect the presence, absence, and/or level of two BV-related bacteria (e.g., BVAB-2 and Megasphaera-1). Such combination may, in some embodiments, reduce the amount of reagent needed to conduct the experiment as well as provide an accurate qualitative metric upon which a BV determination can be assessed.

Without being bound any particular theory, it is believed that the use of combined markers may increase the sensitivity and specificity of the assay. In some embodiments, separate fluorescence channels are used to detect the presence, absence and/or level of each of Lactobacillus spp. (for example L. crispatus, L. jensenii, and L. gasseri), G. vaginalis, and Atopobium vaginae, and a single fluorescence channel is used to detect the presence, absence, and/or level of BVAB-2, Megasphaera-1, Eggerthella spp., and Prevotella spp.

Oligonucleotides (for example amplification primers and probes) that are capable of specifically hybridizing (e.g., under standard nucleic acid amplification conditions, e.g., standard PCR conditions, and/or stringent hybridization conditions) to a target gene region, or complement thereof, in VVC-associated Candida species, L. crispatus, L. jensenii, L. gasseri, G. vaginalis, Atopobium vaginae, Megasphaera Type 1 (Megasphaera-1), and BVAB-2 are provided. Amplification of the target gene region of an organism in a sample (e.g., a vaginal swab sample) can, in some embodiments, be indicative of the presence, absence, and/or level of the organism in the sample.

The target gene region can vary. In some embodiments, oligonucleotides (e.g., amplification primers and probes) that are capable of specifically hybridizing (e.g., under standard nucleic acid amplification conditions, e.g., standard PCR conditions, and/or stringent hybridization conditions) to a gene region encoding 16S ribosomal RNA (16S rRNA) in an organism is provided. In some embodiments, the organism is Gardnerella vaginalis. In some embodiments, the organism is BVAB-2. In some embodiments, the organism is Megasphaera type 1. Examples of oligonucleotides capable of specifically hybridizing to the 16s rRNA gene region in G. vaginalis, Megasphaera-1 and BVAB-2 include but are not limited to SEQ ID NOs: 11-13 as provided in Table I, SEQ ID NOs: 23-29 as provided in Table III, and 30-37 as provided in Table IV, respectively.

In some embodiments, the 23s rRNA gene is used as the target gene for the DNA amplification to detect the presence, absence and/or level of Gardnerella vaginalis, Atopobium vaginae, and Lactobacillus spp. in the sample. Examples of oligonucleotides capable of specifically hybridizing to the 23s RNA gene region in G. vaginalis, A. vaginae and Lactobacillus spp. include but are not limited to SEQ ID NOs: 5-10 as provided in Table I, SEQ ID NOs: 14-16 as provided in Table II, and SEQ ID NOs: 38-43 as provided in Table V, respectively.

In some embodiments, oligonucleotides (e.g., amplification primers and probes) that are capable of specifically hybridizing (e.g., under standard nucleic acid amplification conditions, e.g., standard PCR conditions, and/or stringent hybridization conditions) to a gene region encoding the elongation factor Tu protein (tuf gene) in G. vaginalis and tufA gene in A. vaginae are provided. In some embodiments, the tuf (or tufA) gene is used as the target gene for the DNA amplification to detect the presence, absence and/or level of G. vaginalis and A. vaginae in the sample. In some embodiments, primers and probes that can specifically bind to the tuf gene region of G. vaginalis and tufA gene region of A. vaginae are used in detection of the presence, absence and/or level of G. vaginalis and A. vaginae in a biological sample. Examples of oligonucleotides capable of specifically hybridizing to the tuf gene region in G. vaginalis include, but are not limited to SEQ ID NOs: 1-4 as provided in Table I and examples of oligonucleotides capable of specifically hybridizing to the tufA gene region in A. vaginae include, but are limited to SEQ ID NOs: 17-22 as provided in Table II.

In some embodiments, the ribosomal RNA (rRNA) gene is used as the target gene for the DNA amplification to detect the presence, absence and/or level of VVC-associated Candida species in the sample. In some embodiments, the VVC-associated Candida species comprises C. albicans, C. tropicalis, Candida dubliniensis and C. parapsilosis, (collectively Candida spp.). In some embodiments, the VVC-associated Candida species is Candida krusei. In some embodiments, the VVC-associated Candida species is Candida glabrata. In some embodiments, the VVC-associated Candida species is C. albicans, C. tropicalis, C. dubliniensis C. parapsilosis, or a combination thereof. Examples of oligonucleotides capable of specifically hybridizing to the rRNA gene region in C. glabrata include, but are not limited, SEQ ID NOs: 63-65 as provided in Table VII. Examples of oligonucleotides capable of specifically hybridizing to the rRNA gene region in C. albicans, C. tropicalis, C. dubliniensis and C. parapsilosis (collectively Candida spp.) include, but are not limited, SEQ ID NOs: 48-59 as provided in Table VI. Examples of oligonucleotides capable of specifically hybridizing to the rRNA gene region in C. krusei include, but are not limited, SEQ ID NOs: 60-62 as provided in Table VII.

TABLE I
Primers and Probes for Detection of Gardnerella vaginalis

| Targeted | | | | | SEQ ID |
|---|---|---|---|---|---|
| Gene | Oligo Type | Oligo Name | Sequence | Modification | NO: |
| tuf | Forward | GVAG_tuf_F_1 | AGCTCCCAATCAACACCCCJ | J = t-butylbenzyl | 1 |
| | primer | | | dA | |
| tuf | Reverse | GVAG_tuf_R_1 | CACGGAGAAGAAGACCAGTGTT | K = t-butylbenzyl | 2 |
| | primer | | ATK | dC | |
| tuf | Probe | GVAG_tuf_Pb_1 | <COU>AGACCACCACQCGTCAC | <COU> = Coumarin | 3 |
| | | | CTCTATCGAGACCTTCCSp | -Thr, Q = BHQ2, | |
| | | | | Sp = Spacer-C3 | |
| tuf | Forward | GVAG_tuf_F_2 | CCCAATCAACACCCCAGTTGAGJ | J = t-butylbenzyl | 4 |
| | primer | | | dA | |
| 23s rRNA | Forward | GVAG_23s_F_4 | CTGTAAGGCTGATTCCGGTGATK | K = t-butylbenzyl | 5 |
| | primer | | | dC | |
| 23s rRNA | Reverse | GVAG_23s_R_4 | CGAGTTCCTTGGCCAGGATTK | K = t-butylbenzyl | 6 |
| | primer | | | dC | |
| 23s rRNA | Probe | GVAG_23s_Pb_4 | <COU>CAACGTGAGGQTGTGAT | <COU> = Coumarin | 7 |
| | | | TCCGCTCGTACCCSp | -Thr, Q = BHQ2, | |
| | | | | Sp = Spacer-C3 | |
| 23s rRNA | Forward | GVAG_23s_F_6 | AGTGACGCGGGTAGGTAGK | K = t-butylbenzyl | 8 |
| | primer | | | dC | |
| 23s rRNA | Reverse | GVAG_23s_R_5 | GCAAGCAGGATCACCGGAATCJ | J = t-butylbenzyl | 9 |
| | primer | | | dA | |
| 23s rRNA | Probe | GVAG_23s_Pb_5 | <COU>CTGAGGCGTGQATGGGT | <COU> = Coumarin | 10 |
| | | | AGGCTGTAAGGCSp | -Thr, Q = BHQ2, | |
| | | | | Sp = Spacer-C3 | |
| 16s rRNA | Forward | GVAG_F1 | TGCTCCAGAATAGCTCTTGGJ | J = t-butylbenzyl | 11 |
| | primer | | | dA | |
| 16s rRNA | Reverse | GVAG_R1 | GCCACTAAACACTTTCCCAACJ | J = t-butylbenzyl | 12 |
| | primer | | | dA | |
| 16s rRNA | Probe | GVAG_16s_Pb_1 | <COU>TGCTGGATGCQTCCAACT | <COU> = Coumarin | 13 |
| | | | TGACGCATGSp | -Thr, Q = BHQ2, | |
| | | | | Sp = Spacer-C3 | |

TABLE II
Primers and Probes for Detection of Atopobium vaginae

| Targeted | | | | | SEQ ID |
|---|---|---|---|---|---|
| Gene | Oligo Type | Oligo Name | Sequence | Modification | NO: |
| 23s rRNA | Forward | AVAG_23s_F_1 | GCTGAGTCCGGTTGGGATAJ | J = t-butylbenzyl | 14 |
| | primer | | | dA | |
| 23s rRNA | Reverse | AVAG_23s_R_1 | TTGTCTACAAG<Dz_dG>GGTCT | J = t-butylbenzyl | 15 |
| | primer | | TACCAJ | dA, <Dz_dG> = 7- | |
| | | | | deaza-dG | |
| 23s rRNA | Probe | AVAG_23s_Pb_1 | <HEX>CGGGAAQGCCCATCCCA | <HEX> = HEX-Thr, | 16 |
| | | | AGATGAGTTTTCCTSp | Q = BHQ2, | |
| | | | | Sp = Spacer-C3 | |
| 23s rRNA | Probe | AVAG_23s_Pb_2_ | <HEX>AAGCATQCTAAGCGGGA | <HEX> = HEX-Thr, | 66 |
| | | H_Q6 | AGCCCATCCCASp | Q = BHQ2, | |
| | | | | Sp = Spacer-C3 | |
| 23s rRNA | Forward | AVAG_23S_F_5 | GCCCCAGTCAAACTACCCJ | J = t-butylbenzyl | 67 |
| | primer | | | dA | |
| 23s rRNA | Reverse | AVAG_23S_R_5 | TG<Dz_dG>GAGACTGTGAACTG | J = t-butylbenzyl | 68 |
| | primer | | GAGJ | dA, <Dz_dG> = 7- | |
| | | | | deaza-dG | |
| 23s rRNA | Probe | AVAG_23S_Pb_5 | <HEX>AAG<Dz_dG>GTTQAGGA | <HEX> = HEX-Thr, | 69 |
| | | | TGCCGAAACGTCGAG<Dz_dG>G | Q = BHQ2, | |
| | | | TSp | <Dz_dG> = 7- | |
| | | | | deaza-dG | |
| | | | | Sp = Spacer-C3 | |
| 23s rRNA | Forward | AVAG_23S_F_3 | CGGTCCCCTTCCCGGATTJ | J = t-butylbenzyl | 70 |
| | primer | | | dA | |
| 23s rRNA | Reverse | AVAG_23S_R_6 | CGTGAACCTTTACTGCAGCTTGJ | J = t-butylbenzyl | 71 |
| | primer | | | dA | |
| tufA | Forward | AVAG_tufA_F_2 | GCCTGGCGACCACGTAJ | J = t-butylbenzyl | 17 |
| | primer | | | dA | |
| tufA | Reverse | AVAG_tufA_R_2 | TGTGGCCACCTTCACGAATJ | J = t-butylbenzyl | 18 |
| | primer | | | dA | |
| tufA | Probe | AVAG_tufA_Pb_ | <HEX>ATTCACCCQCATTGCTAT | <HEX> = HEX-Thr, | 19 |
| | | 2 | GGAGGAAGGTCTTAAGTTCGSp | Q = BHQ2, | |
| | | | | Sp = Spacer-C3 | |
| tufA | Forward | AVAG_tufA_F_3 | CACTAAGGTTCTCTCCGAGCAJ | J = t-butylbenzyl | 20 |
| | primer | | | dA | |
| tufA | Reverse | AVAG_tufA_R_3 | TCAATGTGAGCAACGTTAATGGT | J = t-butylbenzyl | 21 |
| | primer | | AJ | dA | |
| tufA | Probe | AVAG_tufA_Pb_ | <HEX>CGACAAQGGCTCCTGAA | <HEX> = HEX-Thr, | 22 |
| | | 3 | GAGCGTCAACGSp | Q = BHQ2, | |
| | | | | Sp = Spacer-C3 | |
| tufA | Probe | AVAG_tufA_Pb_ | <HEX>AGGCTCCQTGAAGAGCG | <HEX> = HEX-Thr, | 72 |
| | | 4 | TCAACGTGGTSp | Q = BHQ2, | |
| | | | | Sp = Spacer-C3 | |

TABLE III
Primers and Probes for Detection of Megasphaera type 1

| Targeted | | | | | SEQ ID |
|---|---|---|---|---|---|
| Gene | Oligo Type | Oligo Name | Sequence | Modification | NO: |
| 16s rRNA | Forward | MEGA1_16s_F_2 | CCGCGTGAGTGAAGAAGGTK | K = t-butylbenzyl | 23 |
| | primer | | | dC | |
| 16s rRNA | Reverse | MEGA1_16s_R_2 | GGTACCGTCACGGACGGATAK | K = t-butylbenzyl | 24 |
| | primer | | | dC | |
| 16s rRNA | Probe | MEGA1_16s_Pb_ | <HEX>AGCTCTQGTTATACGGGA | <HEX> = HEX-Thr, | 25 |
| | | 2 | CGAAAAAGACGGATGCCASp | Q = BHQ2, | |
| | | | | Sp = Spacer-C3 | |
| 16s rRNA | Forward | MEGA1_16s_F_4 | CCGCAATGGACGAAAGTCTGJ | J = t-butylbenzyl | 26 |
| | primer | | | dA | |
| 16s rRNA | Forward | MEGA1_16s_F_6 | TCTTCCGCAATGGACGAAAGTK | K = t-butylbenzyl | 27 |
| | primer | | | dC | |
| 16s rRNA | Reverse | MEGA1_16s_R_6 | GCATCCGTCTTTTTCGTCCCGTAT | J = t-butylbenzyl | 28 |
| | primer | | J | dA | |
| 16s rRNA | Probe | MEGA1_16s_Pb_ | <HEX>CAACGCCGCQGTGAGTG | <HEX> = HEX-Thr, | 29 |
| | | 6 | AAGAAGGTCTTCGGSp | Q = BHQ2, | |
| | | | | Sp = Spacer-C3 | |

TABLE IV
Primers and Probes for Detection of BVAB-2

| Targeted | | | | | SEQ ID |
|---|---|---|---|---|---|
| Gene | Oligo Type | Oligo Name | Sequence | Modification | NO: |
| 16s rRNA | Forward | BVAB2_16s_F_1 | CCCGCACAAGCAGTGGATTJ | J = t-butylbenzyl | 30 |
| | primer | | | dA | |
| 16s rRNA | Reverse | BVAB2_16s_R_1 | TGTCTCTCTGTCTCCGAAGAGAJ | J = t-butylbenzyl | 31 |
| | primer | | | dA | |
| 16s rRNA | Probe | BVAB2_16s_Pb_ | <HEX>ACCTTACCAGGQACTTGA | <HEX> = HEX-Thr, | 32 |
| | | 1 | CATCCTCTGACGATTCASp | Q = BHQ2, | |
| | | | | Sp = Spacer-C3 | |
| 16s rRNA | Forward | BVAB2_16s_F_3 | AGTGTGATGTTTAAATCCAAGGC | J = t-butylbenzyl | 33 |
| | primer | | TTAJ | dA | |
| 16s rRNA | Reverse | BVAB2_16s_R_3 | GTGTTCCTCCTAATATCTACGCA | J = t-butylbenzyl | 34 |
| | primer | | TTTTJ | dA | |
| 16s rRNA | Probe | BVAB2_16s_Pb_ | <HEX>CAAACTGTTTAQGCTTGA | <HEX> = HEX-Thr, | 35 |
| | | 3 | GTGCTGGAGAGGATAGTGGSp | Q = BHQ2, | |
| | | | | Sp = Spacer-C3 | |
| 16s rRNA | Reverse | BVAB2_16s_R_4 | GTTCCTCCTAATATCTACGCATTT | K = t-butylbenzyl | 36 |
| | primer | | TACK | dC | |
| 16s rRNA | Probe | BVAB2_16s_Pb_ | <HEX>TCATTACQAAACTGTTTA | <HEX> = HEX-Thr, | 37 |
| | | 4 | GCTTGAGTGCTGGAGAGGATAG | Q = BHQ2, | |
| | | | TSp | Sp = Spacer-C3 | |

TABLE V
Primers and Probes for Detection of Lactobacillus spp.

| Targeted | | | | | SEQ ID |
|---|---|---|---|---|---|
| Gene | Oligo Type | Oligo Name | Sequence | Modification | NO: |
| 23s rRNA | Forward | LBS_23s_F_1 | TCAGGGGTTACTGAATCCAGAT | J = t-butylbenzyl | 38 |
| | primer | | AAJ | dA | |
| 23s rRNA | Reverse | LBS_23s_R_1 | ACTTAGCATAGATTTGGGGACCT | J = t-butylbenzyl | 39 |
| | primer | | TJ | dA | |
| 23s rRNA | Probe | LBS_23s_Pb_1 | <FAM>AGTCAGACTQGCGAGTG | <FAM> = FAM_Thr, | 40 |
| | | | ATAAGATCCGTAGTCGAAASp | Q = BHQ2, | |
| | | | | Sp = Spacer-C3 | |
| 23s rRNA | Forward | LBS_23s_F_2 | GTGAGAATCCTATCCGCCGAJ | J = t-butylbenzyl | 41 |
| | primer | | | dA | |
| 23s rRNA | Reverse | LBS_23s_R_2 | CTTGCCTTAGGTCCCGACTTJ | J = t-butylbenzyl | 42 |
| | primer | | | dA | |
| 23s rRNA | Probe | LBS_23s_Pb_2 | <FAM>TTCCTGGQGGCAGGCTC | <FAM> = FAM_Thr, | 43 |
| | | | GTCCGCSp | Q = BHQ2, | |
| | | | | Sp = Spacer-C3 | |
| D-LDH | Forward | LBS_LDH_F_1 | GCTCGTGTATTACGTCAAGACAJ | J = t-butylbenzyl | 44 |
| | primer | | | dA | |
| D-LDH | Reverse | LBS_LDH_R_2 | GAAGATATCGTAAGCAATAACC | K = t-butylbenzyl | 45 |
| | primer | | TTTGK | dC | |
| D-LDH | Probe | LBS_LDH_Pb_1 | <FAM>TCGTGAQCCAAGTTGTT | <FAM> = FAM_Thr | 46 |
| | | | GGTGTTGTAGGTACTGGTCASp | Q = BHQ2, | |
| | | | | Sp = Spacer-C3 | |
| D-LDH | Reverse | LBS_LDH_R_1 | CCTTTGCGCCGAAACCTTCK | K = t-butylbenzyl | 47 |
| | primer | | | dC | |
| D-LDH | Reverse | LBS_LDH_R_3 | CATAAATACTTGACCAATGTGAC | J = t-butylbenzyl | 73 |
| | primer | | CAGTJ | dA | |
| D-LDH | Probe | LBS_LDH_PB_3 | <FAM>TGAAGTTCQGTGACCAA | <FAM> = FAM_Thr, | 74 |
| | | | GTTGTTGGTGTTGTAGGTACSp | Q = BHQ2, | |
| | | | | Sp = Spacer-C3 | |

TABLE VI
Primers and Probes for Detection of Candida spp.

| Targeted | | | | | SEQ ID |
|---|---|---|---|---|---|
| Gene | Oligo Type | Oligo Name | Sequence | Modification | NO: |
| rRNA | Forward | CVS_rRNA_F_1 | CAACGGATCTCTTGGTTCTCGCA | K = t-butylbenzyl | 48 |
| | primer | | TK | dC | |
| rRNA | Reverse | CVS_rRNA_R_1 | GGGCGCAATGTGCGTTCAJ | J = t-butylbenzyl | 49 |
| | primer | | | dA | |
| rRNA | Probe | CVS_rRNA_Pb_1 | <JA>CTGCAATTCAQTATTACGTA | <JA> = JA270-Thr, | 50 |
| | | | TCGCATTTCGCTGCGTTSp | Q = BHQ2, | |
| | | | | Sp = Spacer-C3 | |
| rRNA | Forward | CVS_rRNA_F_2 | GCGAGAGACCGATAGCGAACJ | J = t-butylbenzyl | 51 |
| | primer | | | dA | |
| rRNA | Reverse | CVS_rRNA_R_2 | GCCCTTCCCTTTCAACAATTTCAK | K = t-butylbenzyl | 52 |
| | primer | | | dC | |
| rRNA | Probe | CVS_rRNA_Pb_2 | <JA>TTTCACTCTCQTTTTCAAAG | <JA> = JA270-Thr, | 53 |
| | | | TTCTTTTCATCTTTCCATCACTGT | Q = BHQ2, | |
| | | | ACSp | Sp = Spacer-C3 | |
| rRNA | Probe | CVS_rRNA_Pb_3 | <JA>TTTCACTCTQCTTTTCAAAG | <JA> = JA270-Thr, | 75 |
| | | | TGCTTTTCATCTTTCCTTCACAGT | Q = BHQ2, | |
| | | | ACSp | Sp = Spacer-C3 | |
| 25s rRNA | Forward | CAPT_rRNA_F_1 | AATGCGATACGTAATATGAATTG | K = t-butylbenzyl | 54 |
| | primer | | CAGATATTK | dC | |
| 25s rRNA | Reverse | CAPT_rRNA_R_1 | TTTGAGGGAGAAATGACGCTCA | J = t-butylbenzyl | 55 |
| | primer | | AJ | dA | |
| 25s rRNA | Probe | CAPT_rRNA_Pb_ | <JA>ATCGAATCTTQTGAACGCA | <JA> = JA270-Thr, | 56 |
| | | 1 | CATTGCGCCCTTTGGSp | Q = BHQ2, | |
| | | | | Sp = Spacer-C3 | |
| 25s rRNA | Forward | CAPT_rRNA_F_6 | AAGAAACCAACAGGGATTGCCT | J = t-butylbenzyl | 57 |
| | primer | | TAGTJ | dA | |
| 25s rRNA | Reverse | CAPT_rRNA_R_6 | TCATCTCATCGCACGGGATTCTCJ | J = t-butylbenzyl | 58 |
| | primer | | | dA | |
| 25s rRNA | Probe | CAPT_rRNA_Pb_ | <JA>AGTTGTAATTQTGAAGAAG | <JA> = JA270-Thr, | 59 |
| | | 6 | GTATCTTTGGGTCTGGCTCTTGT | Q = BHQ2, | |
| | | | CTASp | Sp = Spacer-C3 | |
| 25s rRNA | Forward | CAPT_rRNA_F_2 | AGTCCGAGTTGTAATTTGAAGA | K = t-butylbenzyl | 76 |
| | primer | | AGGTATK | dC | |
| 25s rRNA | Reverse | CAPT_rRNA_R_2 | TGCATTCCCAAACAACTCGACTK | K = t-butylbenzyl | 77 |
| | primer | | | dC | |
| 25s rRNA | Probe | CAPT_rRNA_Pb_ | <JA>ACGTCACAGAQGGGTGAG | <JA> = JA270-Thr, | 78 |
| | | 2 | AATCCCGTGCGSp | Q = BHQ2, | |
| | | | | Sp = Spacer-C3 | |

TABLE VII
Primers and Probe for Detection of Candida krusei and Candida glabrata

| Targeted | | | | | SEQ ID |
|---|---|---|---|---|---|
| Gene | Oligo Type | Oligo Name | Sequence | Modification | NO: |
| C. krusei | Forward | KRUS_rRNA_F_1 | TCGTGAATCATCGAGTTCTTGAJ | J = t-butylbenzyl | 60 |
| rRNA ITS | primer | | | dA | |
| C. krusei | Reverse | KRUS_rRNA_R_1 | ACACGTCGTCCGCTCK | K = t-butylbenzyl | 61 |
| rRNA ITS | primer | | | dC | |
| C. krusei | Probe | KRUS_rRNA_Pb_ | <JA>TTTGAGCGTCQGTTTCCATC | <JA> = JA270-Thr, | 62 |
| rRNA ITS | | 1 | TTGCGCGSp | Q = BHQ2, | |
| | | | | Sp = Spacer-C3 | |
| C. krusei | Forward | KRUS_rRNA_F_3 | GGTAATTCCAGCTCCAATAGCGT | J = t-butylbenzyl | 79 |
| 18s rRNA | primer | | J | dA | |
| C. krusei | Reverse | KRUS_rRNA_R_3 | GCCTGCTTTGAACACTCTAATTT | K = t-butylbenzyl | 80 |
| 18s rRNA | primer | | CK | dC | |
| C. krusei | Probe | KRUS_rRNA_PB_ | <JA>TAAGCACTQGTTGCGGCCG | <JA> = JA270-Thr, | 81 |
| 18s rRNA | | 3 | GGTCTTTCCSp | Q = BHQ2, | |
| | | | | Sp = Spacer-C3 | |
| C. krusei | Forward | KRUS_rRNA_F_4 | AGTTGTTGCAGTTAAAAAGCTCG | J = t-butylbenzyl | 82 |
| 18s rRNA | primer | | TJ | dA | |
| C. krusei | Reverse | KRUS_rRNA_R_4 | GCGTCCTATTCTATTATTCCATGC | K = t-butylbenzyl | 83 |
| 18s rRNA | primer | | TAATATATK | dC | |
| C. glabrata | Forward | GLAB_rRNA_F_1 | AAGTAAACTCCACTGTGTGTAGT | J = t-butylbenzyl | 63 |
| rRNA ITS | primer | | AJ | dA | |
| C. glabrata | Reverse | GLAB_rRNA_R_1 | AGTGTGCGTGGATCTCTCTJ | J = t-butylbenzyl | 64 |
| rRNA ITS | primer | | | dA | |
| C. glabrata | Probe | GLAB_rRNA_Pb_ | <JA>AGTGTCGAGTQCGTGTGAT | <JA> = JA270-Thr, | 65 |
| rRNA ITS | | 1 | AAAACACCTCCTTTGGSp | Q = BHQ2, | |
| | | | | Sp = Spacer-C3 | |

TABLE VIII
Primers and Probes for Detection of Eggerthella spp.

| Targeted | | | | | SEQ ID |
|---|---|---|---|---|---|
| Gene | Oligo Type | Oligo Name | Sequence | Modification | NO: |
| 16s rRNA | Forward | Egg_16s_F_1 | AACTG<Dz_dG>GAGGCTCGAGT | K = t-butylbenzyl | 84 |
| | primer | | TK | dC, <Dz_dG> = 7- | |
| | | | | deaza-dG | |
| 16s rRNA | Reverse | Egg_16s_R_1 | CCATCGGTGTTCTTCCCGATATK | K = t-butylbenzyl | 85 |
| | primer | | | dC | |
| 16s rRNA | Probe | Egg_16s_Pb_1 | <HEX>AGGCAGQGCGGAATTCC | <HEX> = HEX-Thr, | 86 |
| | | | CGGTGTAGCSp | Q = BHQ2, | |
| | | | | Sp = Spacer-C3 | |
| 16s rRNA | Forward | Egg_16s_F_2 | GGAGGCTCGAGTTCGGTAGJ | J = t-butylbenzyl | 87 |
| | primer | | | dA | |
| 16s rRNA | Reverse | Egg_16s_R_2 | CCATCGGTGTTCTTCCCGATJ | J = t-butylbenzyl | 88 |
| | primer | | | dA | |
| 16s rRNA | Probe | Egg_16s_Pb_2 | <HEX>ACACCGGQGAATTCCGCC | <HEX> = HEX-Thr, | 89 |
| | | | TGCCTCSp | Q = BHQ2, | |
| | | | | Sp = Spacer-C3 | |

TABLE IX
Primers and Probes for Detection of Prevotella spp.

| Targeted | | | | | SEQ ID |
|---|---|---|---|---|---|
| Gene | Oligo Type | Oligo Name | Sequence | Modification | NO: |
| 16s rRNA | Forward | Prev_16s_F_1 | GGATG<Dz_dG>GGATGCGTCT | J = t-butylbenzyl | 90 |
| | primer | | GJ | dA | |
| 16s rRNA | Reverse | Prev_16s_R_1 | GTTTGGACCGTGTCTCAGTTK | K = t-butylbenzyl | 91 |
| | primer | | dC | dC | |
| 16s rRNA | Reverse | Prev_16s_R_2 | CCTCCCGTAGGAGTTTGGAK | K = t-butylbenzyl | 92 |
| | primer | | | dC | |
| 16s rRNA | Reverse | Prev_16s_R_3 | GCCTCCCGTAGGAGTTTGGJ | J = t-butylbenzyl | 93 |
| | primer | | | dA | |
| 16s rRNA | Probe | Prev_16s_Pb_2 | <HEX>TCAGTAGQGGGTTCTGA | <HEX> = HEX-Thr, | 94 |
| | | | GAGGAAGGTCCCCSp | Q = BHQ2, | |
| | | | | Sp = Spacer-C3 | |
| 16s rRNA | Forward | Prev_16s_F_4 | TCAAATCAGCACGGCCCTTJ | J = t-butylbenzyl | 95 |
| | primer | | | dA | |
| 16s rRNA | Forward | Prev_16s_F_5 | CCACCGTAAGGTGTGAGGAJ | J = t-butylbenzyl | 96 |
| | primer | | | dA | |
| 16s rRNA | Forward | Prev_16s_F_6 | CCACCGCAAGGTGCGAGGAJ | J = t-butylbenzyl | 97 |
| | primer | | | dA | |
| 16s rRNA | Reverse | Prev_16s_R_4 | GCACCCCCGGCTTTK | K = t-butylbenzyl | 98 |
| | primer | | | dC | |
| 16s rRNA | Reverse | Prev_16s_R_5 | GGCACCCCCGGCTTTK | K = t-butylbenzyl | 99 |
| | primer | | | dC | |
| 16s rRNA | Reverse | Prev_16s_R_6 | CATGGCTGATGCGCGATTJ | J = t-butylbenzyl | 100 |
| | primer | | | dA | |
| 16s rRNA | Probe | Prev_16s_Pb_4 | <HEX>ACCCCACQGAAGCTGGA | <HEX> = HEX-Thr, | 101 |
| | | | TTCGCTAGTAATCGCSp | Q = BHQ2, | |
| | | | | Sp = Spacer-C3 | |
| 16s rRNA | Probe | Prev_16s_Pb_5 | <HEX><Dz_dG>GTCTGCAQACC | <HEX> = HEX-Thr, | 102 |
| | | | CGACCCCACGAAGCTGGASp | <Dz_dG> = 7- | |
| | | | | deaza-dG, Q = | |
| | | | | BHQ2, Sp = Spacer- | |
| | | | | C3 | |

In one embodiment, the above described sets of primers and probes are used in order to provide for detection of bacteria and Candida species associated with vaginosis in a biological sample suspected of containing such bacteria and Candida species. The sets of primers and probes may comprise or consist of the primers and probes specific for the nucleic acid sequence of the respective bacteria and Candida target gene comprising or consisting of the nucleic acid sequences of SEQ ID NOs: 1-102. In another embodiment, the primers and probes for the target gene comprise or consist of a functionally active variant of any of the primers and probes of SEQ ID NOs: 1-102.

A functionally active variant of any of the primers and/or probes of SEQ ID NOs: 1-102 may be identified by using the primers and/or probes in the disclosed methods. A functionally active variant of a primer and/or probe of any of the SEQ ID NOs: 1-102 pertains to a primer and/or probe which provide a similar or higher specificity and sensitivity in the described method or kit as compared to the respective sequence of SEQ ID NOs: 1-102.

The variant may, e.g., vary from the sequence of SEQ ID NOs: 1-102 by one or more nucleotide additions, deletions or substitutions such as one or more nucleotide additions, deletions or substitutions at the 5′ end and/or the 3′ end of the respective sequence of SEQ ID NOs: 1-102. As detailed above, a primer (and/or probe) may be chemically modified, i.e., a primer and/or probe may comprise a modified nucleotide or a non-nucleotide compound. A probe (or a primer) is then a modified oligonucleotide. “Modified nucleotides” (or “nucleotide analogs”) differ from a natural “nucleotide” by some modification but still consist of a base or base-like compound, a pentofuranosyl sugar or a pentofuranosyl sugar-like compound, a phosphate portion or phosphate-like portion, or combinations thereof. For example, a “label” may be attached to the base portion of a “nucleotide” whereby a “modified nucleotide” is obtained. A natural base in a “nucleotide” may also be replaced by, e.g., a 7-deazapurine whereby a “modified nucleotide” is obtained as well. The terms “modified nucleotide” or “nucleotide analog” are used interchangeably in the present application. A “modified nucleoside” (or “nucleoside analog”) differs from a natural nucleoside by some modification in the manner as outlined above for a “modified nucleotide” (or a “nucleotide analog”).

Oligonucleotides including modified oligonucleotides and oligonucleotide analogs that amplify a nucleic acid molecule encoding the target genes can be designed using, for example, a computer program such as OLIGO (Molecular Biology Insights Inc., Cascade, Colo.). Important features when designing oligonucleotides to be used as amplification primers include, but are not limited to, an appropriate size amplification product to facilitate detection (e.g., by electrophoresis), similar melting temperatures for the members of a pair of primers, and the length of each primer (i.e., the primers need to be long enough to anneal with sequence-specificity and to initiate synthesis but not so long that fidelity is reduced during oligonucleotide synthesis). Typically, oligonucleotide primers are 8 to 50 nucleotides in length (e.g., 8, 10, 12, 14, 16, 18, 20, 22, 24, 26, 28, 30, 32, 34, 36, 38, 40, 42, 44, 46, 48, or 50 nucleotides in length). In some embodiments oligonucleotide primers are 40 or fewer nucleotides in length.

In addition to a set of primers, the methods may use one or more probes in order to detect the presence or absence of target bacterial and Candida species. The term “probe” refers to synthetically or biologically produced nucleic acids (DNA or RNA), which by design or selection, contain specific nucleotide sequences that allow them to hybridize under defined predetermined stringencies specifically (i.e., preferentially) to “target nucleic acids”, in the present case to a target gene nucleic acid. A “probe” can be referred to as a “detection probe” meaning that it detects the target nucleic acid.

In some embodiments, the described target gene probe can be labeled with at least one fluorescent label. In one embodiment, the target gene probe can be labeled with a donor fluorescent moiety, e.g., a fluorescent dye, and a corresponding acceptor moiety, e.g., a quencher. In one embodiment, the probe comprises or consists of a fluorescent moiety and the nucleic acid sequence comprise or consist of SEQ ID NOs: 3, 7, 10, 13, 16, 19, 22, 25, 29, 32, 35, 37, 40, 43, 46, 50, 53, 56, 59, 62 or 65.

Designing oligonucleotides to be used as probes can be performed in a manner similar to the design of primers. Embodiments may use a single probe or a pair of probes for detection of the amplification product. Depending on the embodiment, the probe(s) use may comprise at least one label and/or at least one quencher moiety. As with the primers, the probes usually have similar melting temperatures, and the length of each probe must be sufficient for sequence-specific hybridization to occur but not so long that fidelity is reduced during synthesis. Oligonucleotide probes are generally 15 to 40 (e.g., 16, 18, 20, 21, 22, 23, 24, or 25) nucleotides in length.

Constructs can include vectors each containing one of target gene primers and probes nucleic acid molecules. Constructs can be used, for example, as control template nucleic acid molecules. Vectors suitable for use are commercially available and/or produced by recombinant nucleic acid technology methods routine in the art. Target gene nucleic acid molecules can be obtained, for example, by chemical synthesis, direct cloning from CA, or by PCR amplification.

Constructs suitable for use in the methods typically include, in addition to the target gene nucleic acid molecules (e.g., a nucleic acid molecule that contains one or more sequences of SEQ ID NOs:1-3), sequences encoding a selectable marker (e.g., an antibiotic resistance gene) for selecting desired constructs and/or transformants, and an origin of replication. The choice of vector systems usually depends upon several factors, including, but not limited to, the choice of host cells, replication efficiency, selectability, inducibility, and the ease of recovery.

Constructs containing target gene nucleic acid molecules can be propagated in a host cell. As used herein, the term host cell is meant to include prokaryotes and eukaryotes such as yeast, plant and animal cells. Prokaryotic hosts may include E. coli, Salmonella typhimurium, Serratia marcescens, and Bacillus subtilis. Eukaryotic hosts include yeasts such as S. cerevisiae, S. pombe, Pichia pastoris, mammalian cells such as COS cells or Chinese hamster ovary (CHO) cells, insect cells, and plant cells such as Arabidopsis thaliana and Nicotiana tabacum. A construct can be introduced into a host cell using any of the techniques commonly known to those of ordinary skill in the art. For example, calcium phosphate precipitation, electroporation, heat shock, lipofection, microinjection, and viral-mediated nucleic acid transfer are common methods for introducing nucleic acids into host cells. In addition, naked DNA can be delivered directly to cells (see, e.g., U.S. Pat. Nos. 5,580,859 and 5,589,466).

### Polymerase Chain Reaction (PCR)

U.S. Pat. Nos. 4,683,202, 4,683,195, 4,800,159, and 4,965,188 disclose conventional PCR techniques. PCR typically employs two oligonucleotide primers that bind to a selected nucleic acid template (e.g., DNA or RNA). Primers useful in some embodiments include oligonucleotides capable of acting as points of initiation of nucleic acid synthesis within the described target NG gene nucleic acid sequences. A primer can be purified from a restriction digest by conventional methods, or it can be produced synthetically. The primer is preferably single-stranded for maximum efficiency in amplification, but the primer can be double-stranded. Double-stranded primers are first denatured, i.e., treated to separate the strands. One method of denaturing double stranded nucleic acids is by heating.

If the template nucleic acid is double-stranded, it is necessary to separate the two strands before it can be used as a template in PCR. Strand separation can be accomplished by any suitable denaturing method including physical, chemical or enzymatic means. One method of separating the nucleic acid strands involves heating the nucleic acid until it is predominately denatured (e.g., greater than 50%, 60%, 70%, 80%, 90% or 95% denatured). The heating conditions necessary for denaturing template nucleic acid will depend, e.g., on the buffer salt concentration and the length and nucleotide composition of the nucleic acids being denatured, but typically range from about 90° C. to about 105° C. for a time depending on features of the reaction such as temperature and the nucleic acid length. Denaturation is typically performed for about 30 sec to 4 min (e.g., 1 min to 2 min 30 sec, or 1.5 min).

If the double-stranded template nucleic acid is denatured by heat, the reaction mixture is allowed to cool to a temperature that promotes annealing of each primer to its target sequence on the described target NG gene nucleic acid molecules. The temperature for annealing is usually from about 35° C. to about 65° C. (e.g., about 40° C. to about 60° C.; about 45° C. to about 50° C.). Annealing times can be from about 10 sec to about 1 min (e.g., about 20 sec to about 50 sec; about 30 sec to about 40 sec). The reaction mixture is then adjusted to a temperature at which the activity of the polymerase is promoted or optimized, i.e., a temperature sufficient for extension to occur from the annealed primer to generate products complementary to the template nucleic acid. The temperature should be sufficient to synthesize an extension product from each primer that is annealed to a nucleic acid template, but should not be so high as to denature an extension product from its complementary template (e.g., the temperature for extension generally ranges from about 40° C. to about 80° C. (e.g., about 50° C. to about 70° C.; about 60° C.). Extension times can be from about 10 sec to about 5 min (e.g., about 30 sec to about 4 min; about 1 min to about 3 min; about 1 min 30 sec to about 2 min).

PCR assays can employ nucleic acid such as RNA or DNA (cDNA). The template nucleic acid need not be purified; it may be a minor fraction of a complex mixture, such as nucleic acid contained in human cells. Nucleic acid molecules may be extracted from a biological sample by routine techniques such as those described in Diagnostic Molecular Microbiology: Principles and Applications (Persing et al. (eds), 1993, American Society for Microbiology, Washington D.C.). Nucleic acids can be obtained from any number of sources, such as plasmids, or natural sources including bacteria, yeast, protozoa viruses, organelles, or higher organisms such as plants or animals.

The oligonucleotide primers are combined with PCR reagents under reaction conditions that induce primer extension. For example, chain extension reactions generally include 50 mM KCl, 10 mM Tris-HCl (pH 8.3), 15 mM MgCl₂, 0.001% (w/v) gelatin, 0.5-1.0 μg protodenatured template DNA, 50 pmoles of each oligonucleotide primer, 2.5 U of Taq polymerase, and 10% DMSO). The reactions usually contain 150 to 320 μM each of dATP, dCTP, dTTP, dGTP, or one or more analogs thereof.

The newly synthesized strands form a double-stranded molecule that can be used in the succeeding steps of the reaction. The steps of strand separation, annealing, and elongation can be repeated as often as needed to produce the desired quantity of amplification products corresponding to the target nucleic acid molecules. The limiting factors in the reaction are the amounts of primers, thermostable enzyme, and nucleoside triphosphates present in the reaction. The cycling steps (i.e., denaturation, annealing, and extension) are preferably repeated at least once. For use in detection, the number of cycling steps will depend, e.g., on the nature of the sample. If the sample is a complex mixture of nucleic acids, more cycling steps will be required to amplify the target sequence sufficient for detection. Generally, the cycling steps are repeated at least about 20 times, but may be repeated as many as 40, 60, or even 100 times.

### Fluorescence Resonance Energy Transfer (FRET)

FRET technology (see, for example, U.S. Pat. Nos. 4,996,143, 5,565,322, 5,849,489, and 6,162,603) is based on a concept that when a donor fluorescent moiety and a corresponding acceptor fluorescent moiety are positioned within a certain distance of each other, energy transfer takes place between the two fluorescent moieties that can be visualized or otherwise detected and/or quantitated. The donor typically transfers the energy to the acceptor when the donor is excited by light radiation with a suitable wavelength. The acceptor typically re-emits the transferred energy in the form of light radiation with a different wavelength. In certain systems, non-fluorescent energy can be transferred between donor and acceptor moieties, by way of biomolecules that include substantially non-fluorescent donor moieties (see, for example, U.S. Pat. No. 7,741,467).

In one example, an oligonucleotide probe can contain a donor fluorescent moiety and a corresponding quencher, which may or not be fluorescent, and which dissipates the transferred energy in a form other than light. When the probe is intact, energy transfer typically occurs between the donor and acceptor moieties such that fluorescent emission from the donor fluorescent moiety is quenched the acceptor moiety. During an extension step of a polymerase chain reaction, a probe bound to an amplification product is cleaved by the 5′ to 3′ nuclease activity of, e.g., a Taq Polymerase such that the fluorescent emission of the donor fluorescent moiety is no longer quenched. Exemplary probes for this purpose are described in, e.g., U.S. Pat. Nos. 5,210,015, 5,994,056, and 6,171,785. Commonly used donor-acceptor pairs include the FAM-TAMRA pair. Commonly used quenchers are DABCYL and TAMRA. Commonly used dark quenchers include BlackHole Quenchers™ (BHQ), (Biosearch Technologies, Inc., Novato, Calif.), Iowa Black™, (Integrated DNA Tech., Inc., Coralville, Iowa), BlackBerry™ Quencher 650 (BBQ-650), (Berry & Assoc., Dexter, Mich.).

In another example, two oligonucleotide probes, each containing a fluorescent moiety, can hybridize to an amplification product at particular positions determined by the complementarity of the oligonucleotide probes to the target nucleic acid sequence. Upon hybridization of the oligonucleotide probes to the amplification product nucleic acid at the appropriate positions, a FRET signal is generated. Hybridization temperatures can range from about 35° C. to about 65° C. for about 10 sec to about 1 min.

Fluorescent analysis can be carried out using, for example, a photon counting epifluorescent microscope system (containing the appropriate dichroic mirror and filters for monitoring fluorescent emission at the particular range), a photon counting photomultiplier system, or a fluorimeter. Excitation to initiate energy transfer, or to allow direct detection of a fluorophore, can be carried out with an argon ion laser, a high intensity mercury (Hg) arc lamp, a fiber optic light source, or other high intensity light source appropriately filtered for excitation in the desired range.

As used herein with respect to donor and corresponding acceptor moieties “corresponding” refers to an acceptor fluorescent moiety or a dark quencher having an absorbance spectrum that overlaps the emission spectrum of the donor fluorescent moiety. The wavelength maximum of the emission spectrum of the acceptor fluorescent moiety should be at least 100 nm greater than the wavelength maximum of the excitation spectrum of the donor fluorescent moiety. Accordingly, efficient non-radiative energy transfer can be produced there between.

Fluorescent donor and corresponding acceptor moieties are generally chosen for (a) high efficiency Forster energy transfer; (b) a large final Stokes shift (>100 nm); (c) shift of the emission as far as possible into the red portion of the visible spectrum (>600 nm); and (d) shift of the emission to a higher wavelength than the Raman water fluorescent emission produced by excitation at the donor excitation wavelength. For example, a donor fluorescent moiety can be chosen that has its excitation maximum near a laser line (for example, Helium-Cadmium 442 nm or Argon 488 nm), a high extinction coefficient, a high quantum yield, and a good overlap of its fluorescent emission with the excitation spectrum of the corresponding acceptor fluorescent moiety. A corresponding acceptor fluorescent moiety can be chosen that has a high extinction coefficient, a high quantum yield, a good overlap of its excitation with the emission of the donor fluorescent moiety, and emission in the red part of the visible spectrum (>600 nm).

Representative donor fluorescent moieties that can be used with various acceptor fluorescent moieties in FRET technology include fluorescein, Lucifer Yellow, B-phycoerythrin, 9-acridineisothiocyanate, Lucifer Yellow VS, 4-acetamido-4′-isothio-cyanatostilbene-2,2′-disulfonic acid, 7-diethylamino-3-(4′-isothiocyanatophenyl)-4-methylcoumarin, succinimdyl 1-pyrenebutyrate, and 4-acetamido-4′-isothiocyanatostilbene-2,2′-disulfonic acid derivatives. Representative acceptor fluorescent moieties, depending upon the donor fluorescent moiety used, include LC Red 640, LC Red 705, Cy5, Cy5.5, Lissamine rhodamine B sulfonyl chloride, tetramethyl rhodamine isothiocyanate, rhodamine x isothiocyanate, erythrosine isothiocyanate, fluorescein, diethylenetriamine pentaacetate, or other chelates of Lanthanide ions (e.g., Europium, or Terbium). Donor and acceptor fluorescent moieties can be obtained, for example, from Molecular Probes (Junction City, Oreg.) or Sigma Chemical Co. (St. Louis, Mo.).

The donor and acceptor fluorescent moieties can be attached to the appropriate probe oligonucleotide via a linker arm. The length of each linker arm is important, as the linker arms will affect the distance between the donor and acceptor fluorescent moieties. The length of a linker arm can be the distance in Angstroms (Å) from the nucleotide base to the fluorescent moiety. In general, a linker arm is from about 10 Å to about 25 Å. The linker arm may be of the kind described in WO 84/03285. WO 84/03285 also discloses methods for attaching linker arms to a particular nucleotide base, and also for attaching fluorescent moieties to a linker arm.

An acceptor fluorescent moiety, such as an LC Red 640, can be combined with an oligonucleotide which contains an amino linker (e.g., C6-amino phosphoramidites available from ABI (Foster City, Calif.) or Glen Research (Sterling, Va.)) to produce, for example, LC Red 640-labeled oligonucleotide. Frequently used linkers to couple a donor fluorescent moiety such as fluorescein to an oligonucleotide include thiourea linkers (FITC-derived, for example, fluorescein-CPG's from Glen Research or ChemGene (Ashland, Mass.)), amide-linkers (fluorescein-NHS-ester-derived, such as CX-fluorescein-CPG from BioGenex (San Ramon, Calif.)), or 3′-amino-CPGs that require coupling of a fluorescein-NHS-ester after oligonucleotide synthesis.

### Detection by Real-Time PCR

The present disclosure provides methods for detecting the presence or absence of bacterial and fungal target organisms in a biological or non-biological sample. Methods provided avoid problems of sample contamination, false negatives, and false positives. The methods include performing at least one cycling step that includes amplifying a portion of target nucleic acid molecules from a sample using one or more pairs of primers, and a FRET detecting step. Multiple cycling steps are performed, preferably in a thermocycler. Methods can be performed using the primers and probes to detect the presence of target organisms, and the detection of the target genes indicates the presence of the target organisms in the sample.

As described herein, amplification products can be detected using labeled hybridization probes that take advantage of FRET technology. One FRET format utilizes TaqMan® technology to detect the presence or absence of an amplification product, and hence, the presence or absence of CA. TaqMan® technology utilizes one single-stranded hybridization probe labeled with, e.g., one fluorescent dye and one quencher, which may or may not be fluorescent. When a first fluorescent moiety is excited with light of a suitable wavelength, the absorbed energy is transferred to a second fluorescent moiety or a dark quencher according to the principles of FRET. The second moiety is generally a quencher molecule. During the annealing step of the PCR reaction, the labeled hybridization probe binds to the target DNA (i.e., the amplification product) and is degraded by the 5′ to 3′ nuclease activity of, e.g., the Taq Polymerase during the subsequent elongation phase. As a result, the fluorescent moiety and the quencher moiety become spatially separated from one another. As a consequence, upon excitation of the first fluorescent moiety in the absence of the quencher, the fluorescence emission from the first fluorescent moiety can be detected. By way of example, an ABI PRISM® 7700 Sequence Detection System (Applied Biosystems) uses TaqMan® technology, and is suitable for performing the methods described herein for detecting the presence or absence of NG in the sample.

Molecular beacons in conjunction with FRET can also be used to detect the presence of an amplification product using the real-time PCR methods. Molecular beacon technology uses a hybridization probe labeled with a first fluorescent moiety and a second fluorescent moiety. The second fluorescent moiety is generally a quencher, and the fluorescent labels are typically located at each end of the probe. Molecular beacon technology uses a probe oligonucleotide having sequences that permit secondary structure formation (e.g., a hairpin). As a result of secondary structure formation within the probe, both fluorescent moieties are in spatial proximity when the probe is in solution. After hybridization to the target nucleic acids (i.e., amplification products), the secondary structure of the probe is disrupted and the fluorescent moieties become separated from one another such that after excitation with light of a suitable wavelength, the emission of the first fluorescent moiety can be detected.

Another common format of FRET technology utilizes two hybridization probes. Each probe can be labeled with a different fluorescent moiety and are generally designed to hybridize in close proximity to each other in a target DNA molecule (e.g., an amplification product). A donor fluorescent moiety, for example, fluorescein, is excited at 470 nm by the light source of the LightCycler® Instrument. During FRET, the fluorescein transfers its energy to an acceptor fluorescent moiety such as LightCycler®-Red 640 (LC Red 640) or LightCycler®-Red 705 (LC Red 705). The acceptor fluorescent moiety then emits light of a longer wavelength, which is detected by the optical detection system of the LightCycler® instrument. Efficient FRET can only take place when the fluorescent moieties are in direct local proximity and when the emission spectrum of the donor fluorescent moiety overlaps with the absorption spectrum of the acceptor fluorescent moiety. The intensity of the emitted signal can be correlated with the number of original target DNA molecules (e.g., the number of CA genomes). If amplification of target nucleic acid occurs and an amplification product is produced, the step of hybridizing results in a detectable signal based upon FRET between the members of the pair of probes.

Generally, the presence of FRET indicates the presence of target organism(s) in the sample, and the absence of FRET indicates the absence of target organism(s) in the sample. Inadequate specimen collection, transportation delays, inappropriate transportation conditions, or use of certain collection swabs (calcium alginate or aluminum shaft) are all conditions that can affect the success and/or accuracy of a test result, however. Using the methods disclosed herein, detection of FRET within, e.g., 45 cycling steps is indicative of the presence of the target organism(s).

Representative biological samples that can be used in practicing the methods include, but are not limited to vaginal swabs, fecal specimens, blood specimens, dermal swabs, nasal swabs, wound swabs, blood cultures, skin, and soft tissue infections. Collection and storage methods of biological samples are known to those of skill in the art. Biological samples can be processed (e.g., by nucleic acid extraction methods and/or kits known in the art) to release target nucleic acid or in some cases, the biological sample can be contacted directly with the PCR reaction components and the appropriate oligonucleotides.

Melting curve analysis is an additional step that can be included in a cycling profile. Melting curve analysis is based on the fact that DNA melts at a characteristic temperature called the melting temperature (Tm), which is defined as the temperature at which half of the DNA duplexes have separated into single strands. The melting temperature of a DNA depends primarily upon its nucleotide composition. Thus, DNA molecules rich in G and C nucleotides have a higher Tm than those having an abundance of A and T nucleotides. By detecting the temperature at which signal is lost, the melting temperature of probes can be determined. Similarly, by detecting the temperature at which signal is generated, the annealing temperature of probes can be determined. The melting temperature(s) of the probes from the amplification products can confirm the presence or absence of target organism(s) in the sample.

Within each thermocycler run, control samples can be cycled as well. Positive control samples can amplify target nucleic acid control template (other than described amplification products of target genes) using, for example, control primers and control probes. Positive control samples can also amplify, for example, a plasmid construct containing the target nucleic acid molecules. Such a plasmid control can be amplified internally (e.g., within the sample) or in a separate sample run side-by-side with the patients' samples using the same primers and probe as used for detection of the intended target. Such controls are indicators of the success or failure of the amplification, hybridization, and/or FRET reaction. Each thermocycler run can also include a negative control that, for example, lacks target template DNA. Negative control can measure contamination. This ensures that the system and reagents would not give rise to a false positive signal. Therefore, control reactions can readily determine, for example, the ability of primers to anneal with sequence-specificity and to initiate elongation, as well as the ability of probes to hybridize with sequence-specificity and for FRET to occur.

In an embodiment, the methods include steps to avoid contamination. For example, an enzymatic method utilizing uracil-DNA glycosylase is described in U.S. Pat. Nos. 5,035,996, 5,683,896 and 5,945,313 to reduce or eliminate contamination between one thermocycler run and the next.

Conventional PCR methods in conjunction with FRET technology can be used to practice the methods. In one embodiment, a LightCycler® instrument is used. The following patent applications describe real-time PCR as used in the LightCycler® technology: WO 97/46707, WO 97/46714, and WO 97/46712.

The LightCycler® can be operated using a PC workstation and can utilize a Windows NT operating system. Signals from the samples are obtained as the machine positions the capillaries sequentially over the optical unit. The software can display the fluorescence signals in real-time immediately after each measurement. Fluorescent acquisition time is 10-100 milliseconds (msec). After each cycling step, a quantitative display of fluorescence vs. cycle number can be continually updated for all samples. The data generated can be stored for further analysis.

As an alternative to FRET, an amplification product can be detected using a double-stranded DNA binding dye such as a fluorescent DNA binding dye (e.g., SYBR® Green or SYBR® Gold (Molecular Probes)). Upon interaction with the double-stranded nucleic acid, such fluorescent DNA binding dyes emit a fluorescence signal after excitation with light at a suitable wavelength. A double-stranded DNA binding dye such as a nucleic acid intercalating dye also can be used. When double-stranded DNA binding dyes are used, a melting curve analysis is usually performed for confirmation of the presence of the amplification product.

It is understood that the embodiments of the present disclosure are not limited by the configuration of one or more commercially available instruments.

### Articles of Manufacture/Kits

Embodiments of the present disclosure further provide for articles of manufacture, compositions or kits to detect bacterial and fungal organisms associated with vaginosis. An article of manufacture can include primers and probes used to detect the target genes, together with suitable packaging materials. Compositions can include primers used to amplify the target genes. In certain embodiments compositions can also comprise probes for detecting the target genes. Representative primers and probes for detection of target organism(s) are capable of hybridizing to target nucleic acid molecules. In addition, the kits may also include suitably packaged reagents and materials needed for DNA immobilization, hybridization, and detection, such solid supports, buffers, enzymes, and DNA standards. Methods of designing primers and probes are disclosed herein, and representative examples of primers and probes that amplify and hybridize to target nucleic acid molecules are provided.

Articles of manufacture can also include one or more fluorescent moieties for labeling the probes or, alternatively, the probes supplied with the kit can be labeled. For example, an article of manufacture may include a donor and/or an acceptor fluorescent moiety for labeling the probes. Examples of suitable FRET donor fluorescent moieties and corresponding acceptor fluorescent moieties are provided above.

Articles of manufacture can also contain a package insert or package label having instructions thereon for using the primers and probes to detect target organisms in a sample. Articles of manufacture and compositions may additionally include reagents for carrying out the methods disclosed herein (e.g., buffers, polymerase enzymes, co-factors, or agents to prevent contamination). Such reagents may be specific for one of the commercially available instruments described herein.

Embodiments of the present disclosure will be further described in the following examples, which do not limit the scope of the invention described in the claims.

### EXAMPLES

The following examples, tables and figures are provided to aid the understanding of the subject matter, the true scope of which is set forth in the appended claims. It is understood that modifications can be made in the procedures set forth without departing from the spirit of the invention.

### Example 1 PCR Assay Reagents and Conditions

Real-time PCR detection of target bacteria and Candida species were performed using either the Cobas® 4800 system or the Cobas® 6800/8800 systems platforms (Roche Molecular Systems, Inc., Pleasanton, Calif.). The final concentrations of the amplification reagents are shown below:

TABLE X
PCR Amplification Reagents

| Master Mix Component | Final Conc (50 uL) | |
|---|---|---|
| DMSO | 0-5.4 | % |
| NaN3 | 0.027-0.030 | % |
| Potassium acetate | 120.0 | mM |
| Glycerol | 3.0 | % |
| Tween 20 | 0.02 | % |
| EDTA | 0-43.9 | uM |
| Tricine | 60.0 | mM |
| Aptamer | 0.18-0.22 | uM |
| UNG Enzyme | 5.0-10.0 | U |
| Z05-SP-PZ Polymerase | 30.0-45.0 | U |
| dATP | 400.0-521.70 | uM |
| dCTP | 400.0-521.70 | uM |
| dGTP | 400.0-521.70 | uM |
| dUTP | 800.0-1043.40 | uM |
| Forward primer oligonucleotides | 0.15-0.50 | μM |
| Reverse primer oligonucleotides | 0.15-0.50 | μM |
| Probe oligonucleotides | 0.10 | μM |
| Manganese Acetate | 3.30-3.80 | mM |

The following table shows the typical thermoprofile used for PCR amplification reaction:

TABLE XI
PCR Thermoprofile

| | Target | Acquisition | Hold | Ramp Rate | | |
|---|---|---|---|---|---|---|
| Program Name | (° C.) | Mode | (hh:mm:ss) | (° C./s) | Cycles | Analysis Mode |
| Pre-PCR | 50 | None | 00:02:00 | 4.4 | 1 | None |
| | 94 | None | 00:00:05 | 4.4 | | |
| | 55 | None | 00:02:00 | 2.2 | | |
| | 60 | None | 00:06:00 | 4.4 | | |
| | 65 | None | 00:04:00 | 4.4 | | |
| 1st Measurement | 95 | None | 00:00:05 | 4.4 | 5 | Quantification |
| | 55 | Single | 00:00:30 | 2.2 | | |
| 2nd Measurment | 91 | None | 00:00:05 | 4.4 | 45 | Quantification |
| | 58 | Single | 00:00:25 | 2.2 | | |
| Cooling | 40 | None | 00:02:00 | 2.2 | 1 | None |

The Pre-PCR program comprised initial denaturing and incubation at 55° C., 60° C. and 65° C. for reverse transcription of RNA templates. Incubating at three temperatures combines the advantageous effects that at lower temperatures slightly mismatched target sequences (such as genetic variants of an organism) are also transcribed, while at higher temperatures the formation of RNA secondary structures is suppressed, thus leading to a more efficient transcription. PCR cycling was divided into two measurements, wherein both measurements apply a one-step setup (combining annealing and extension). The first 5 cycles at 55° C. allow for an increased inclusivity by pre-amplifying slightly mismatched target sequences, whereas the 45 cycles of the second measurement provide for an increased specificity by using an annealing/extension temperature of 58° C.

### Example 2 Amplification and Detection of Atopobium vaginae

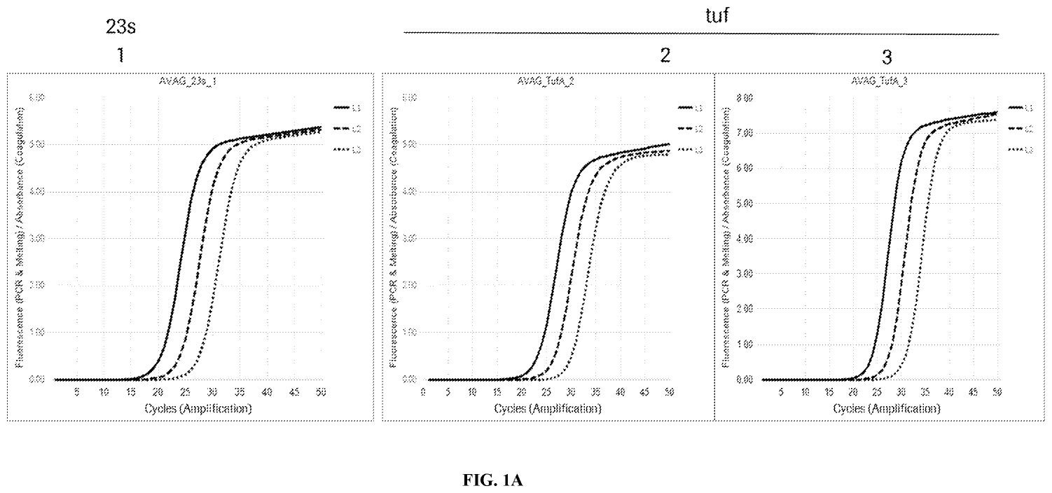

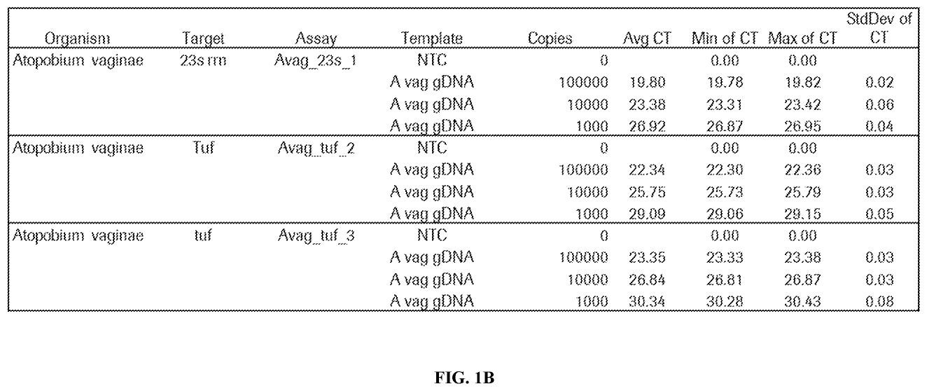

The amplification and detection of the target genes for Atopobium vaginae, 23s ribosomal RNA and tufA, were performed using the conditions described in Example 1. For the AVAG_23s_1 assay the forward primer of SEQ ID NO: 14, the reverse primer of SEQ ID NO: 15 and the probe of SEQ ID NO: 16 were used. For the AVAG_TufA_2 assay, the forward primer of SEQ ID NO: 17, the reverse primer of SEQ ID NO: 18 and the probe of SEQ ID NO: 19 were used. The forward primer of SEQ ID NO: 20, the reverse primer of SEQ ID NO: 21 and the probe of SEQ ID NO: 22 was used in the AVAG_TufA_3 assay. These primers and probes were tested with genomic A. vaginae DNA present at concentrations of 1e5, 1e4 and 1e3 copies per PCR reaction. Results of the experiments are shown as growth curves in FIG. 1A and as calculated Ct values in FIG. 1B. All three assays demonstrated good linearity for the detection of A. vaginae.

### Example 3 Amplification and Detection of Gardnerella vaginalis

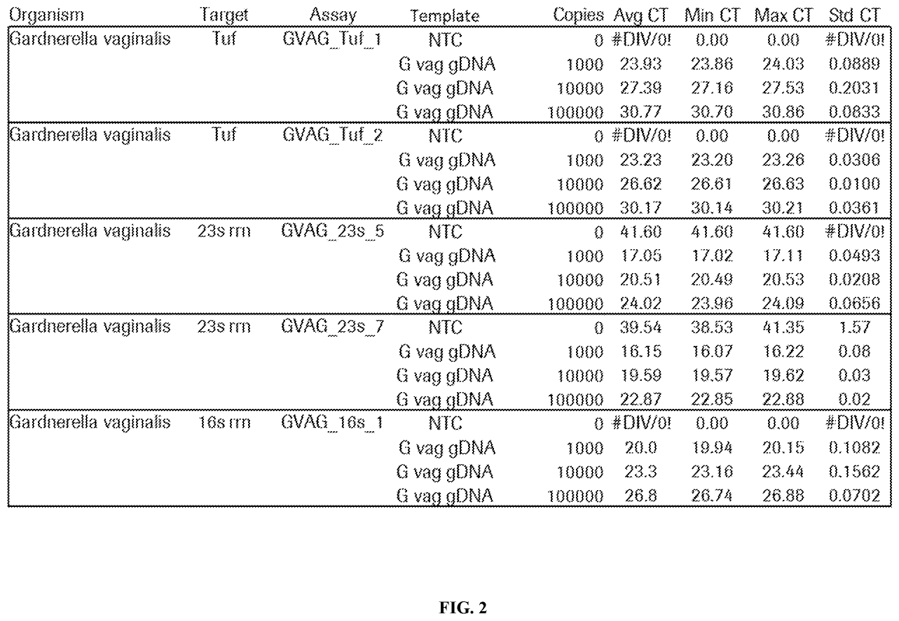

For the amplification and detection of Gardnerella vaginalis, five assays with three target genes were performed. In the GVAG_Tuf_1 assay (forward primer of SEQ ID NO: 1, reverse primer of SEQ ID NO: 2, probe of SEQ ID NO: 3) and the GVAG_Tuf_2 assay (forward primer of SEQ ID NO: 4, reverse primer of SEQ ID NO: 2, probe of SEQ ID NO: 3), the tuf gene was used as the target gene. The 23s rRNA gene was the target for the GVAG_23s_5 assay (forward primer of SEQ ID NO: 5, reverse primer of SEQ ID NO: 6, probe of SEQ ID NO: 7) and for the GVAG_23s_7 assay (forward primer of SEQ ID NO: 8, reverse primer of SEQ ID NO: 9, probe of SEQ ID NO: 10). For the GVAG_16s_1 assay, forward primer of SEQ ID NO: 11, reverse primer of SEQ ID NO: 12 and probe of SEQ ID NO:13 were used to amplify and detect the 16s rRNA gene. These primers and probes were tested with genomic G. vaginalis DNA present at concentrations of 1e5, 1e4 and 1e3 copies per PCR reaction and the results, expressed as Ct values, are shown in FIG. 2.

### Example 4 Amplification and Detection of BVAB-2

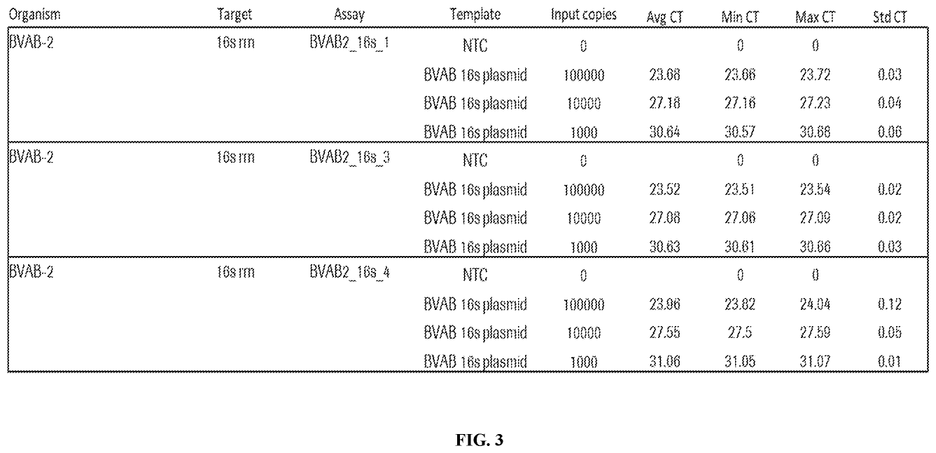

The 16s rRNA gene was used as the target gene for the amplification and detection of BVAB-2 and three assays with the following primers and probes were performed: BVAB2_16s_1: forward primer of SEQ ID NO: 30, reverse primer of SEQ ID NO: 31, probe of SEQ ID NO: 32; BVAB2_16s_3: forward primer of SEQ ID NO: 33, reverse primer of SEQ ID NO: 34, probe of SEQ ID NO: 35; BVAB2_16s_4: forward primer of SEQ ID NO: 33, reverse primer of SEQ ID NO: 36, probe of SEQ ID NO: 37. These assays used 16s rRNA plasmid as template present at concentrations of 1e5, 1e4 and 1e3 copies per PCR reaction and the results, expressed as Ct values, are shown in FIG. 3.

### Example 5 Amplification and Detection of Megasphaera Type 1

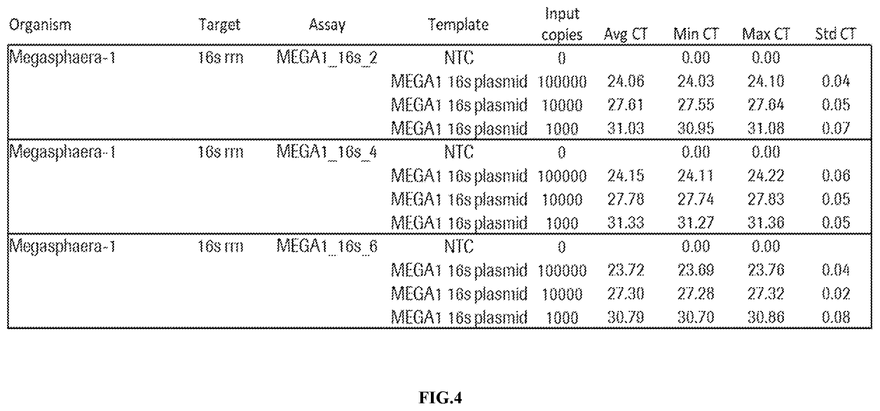

The 16s rRNA gene was used as the target gene for the amplification and detection of Megasphaera type land three assays with the following primers and probes were performed: MEGA1_16s_2: forward primer of SEQ ID NO: 23, reverse primer of SEQ ID NO: 24, probe of SEQ ID NO: 25; MEGA1_16s_4: forward primer of SEQ ID NO: 26, reverse primer of SEQ ID NO: 24, probe of SEQ ID NO: 25; MEGA1_16s_6: forward primer of SEQ ID NO: 27, reverse primer of SEQ ID NO: 28, probe of SEQ ID NO: 29. These assays used 16s rRNA plasmid as template present at concentrations of 1e5, 1e4 and 1e3 copies per PCR reaction and the results, expressed as Ct values, are shown in FIG. 4.

### Example 6 Amplification and Detection of Lactobacillus Spp.

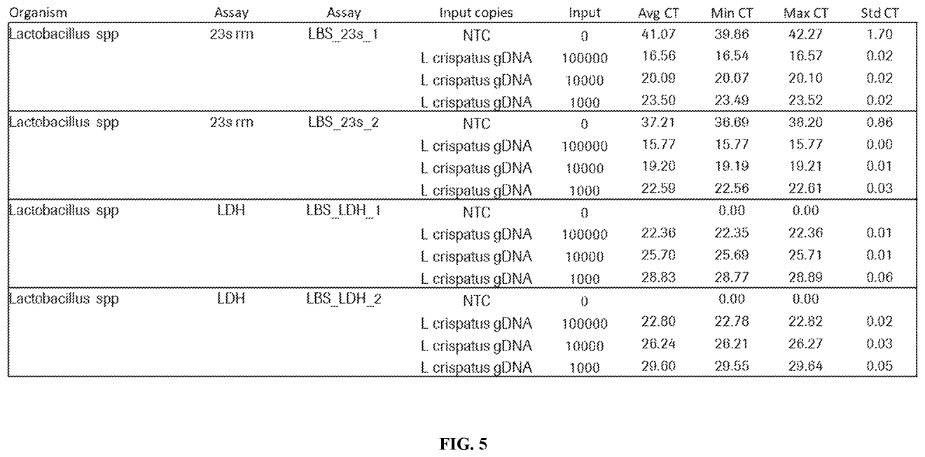

The amplification and detection of Lactobacillus spp. (Lactobacillus crispatus (L. crispatus), Lactobacillus jensenii (L. jensenii), and Lactobacillus gasseri (L. gasseri)) utilized two target genes, 23s ribosomal RNA (23s rRNA) and D-lactate dehydrogenase (LDH) and four assays. In each assay, the following primers and probes were used: LBS_23s_1: forward primer of SEQ ID NO: 38, reverse primer of SEQ ID NO: 39, probe of SEQ ID NO: 40; LBS_23s_2: forward primer of SEQ ID NO: 41, reverse primer of SEQ ID NO: 42, probe of SEQ ID NO: 43; LBS_LDH_1: forward primer of SEQ ID NO: 44, reverse primer of SEQ ID NO: 47, probe of SEQ ID NO: 46; LBS_LDH_2: forward primer of SEQ ID NO: 44, reverse primer of SEQ ID NO: 45, probe of SEQ ID NO: 46. These primers and probes were tested with genomic Lactobacillus DNA present at concentrations of 1e5, 1e4 and 1e3 copies per PCR reaction and the results, expressed as Ct values, are shown in FIG. 5.

### Example 7 Amplification and Detection of Eggerthella Spp.

The 16s ribosomal RNA (16s rRNA) from Eggerthella lenta was used as the target for the DNA amplification and detection of Eggerthella spp. Selection of candidate primer and probe sequences were based on inclusivity against Eggerthella lenta, Eggerthella sinensis, Eggerthella timonensis and unclassified Eggerthella sp. and exclusivity against Atopobium spp., Coriobacterium glomerans, Collinsella vaginalis, Slackia exigua, Olsenella spp., Acetomicrobium faecale, Lactobacillus spp., Bifidobacterium longum, Mobiluncus spp., and Burkholderia. Primer sequences of SEQ ID NOs: 84, 85, 87 and 88 and probe sequences of SEQ ID NOs: 86 and 89 (TABLE VIII) were chosen as a result.

### Example 8 Amplification and Detection of Prevotella Spp.

The 16s ribosomal RNA (16s rRNA) from Prevotella bivia was used as the target for the DNA amplification and detection of Prevotella spp. Selection of candidate primer and probe sequences were based on inclusivity for the following Prevotella species: Prevotella amnii, Prevotella bivia, Prevotella buccalis, Prevotella corporis, Prevotella disiens, Prevotella intermedia. Prevotella melaninogenica, Prevotella nigrescens, Prevotella oris, Prevotella timonensis, Prevotella denticola, Prevotella leoscheii, Prevotella corporis, Prevotella massiliensis, Prevotella bergensis, Prevotella oralis, and Prevotella lascolaii. The candidate primer and probe sequences were also based on exclusivity against Rifkennellaceae, Bacterioidaceae, Porphyromonas, Fusobacterium nucleatum, Sneathia amnii, Mycoplasma genitalium, Lactobacillus spp., Bifidobacterium longum, and Burkholderia. Primer sequences of SEQ ID NOs: 90-93, and 95-100 and probe sequences of SEQ ID NOs: 94, 101-102 (TABLE IX) were chosen as a result.

### Example 9 Amplification and Detection of Candida Spp.

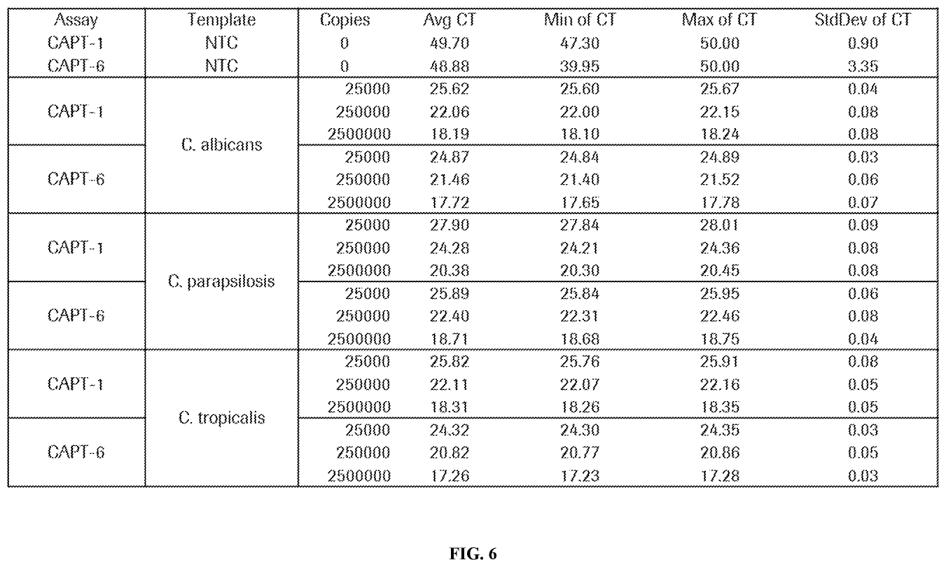

The ribosomal RNA (rRNA) gene is used as the target gene for the DNA amplification to detect the presence, absence and/or level of VVC-associated Candida species in the sample. In the CAPT_rRNA_1 and CAPT_rRNA_6 assays, the VVC-associated Candida species comprises C. albicans, C. parapsilosis, C. dubliniensis and C. tropicalis, (collectively Candida spp.). The CAPT_rRNA_1 assay uses the forward primer of SEQ ID NO: 54, the reverse primer of SEQ ID NO: 55 and the probe of SEQ ID NO: 56. The CAPT_rRNA_6 assay uses the forward primer of SEQ ID NO: 57, the reverse primer of SEQ ID NO: 58 and the probe of SEQ ID NO: 59. Both assays were tested with genomic DNA from C. albicans, C. parapsilosis and C. tropicalis, at concentrations of 2.5e6, 2.5e5 and 2.5e4 copies per PCR reaction, and the results, expressed as Ct values, are shown in FIG. 6.

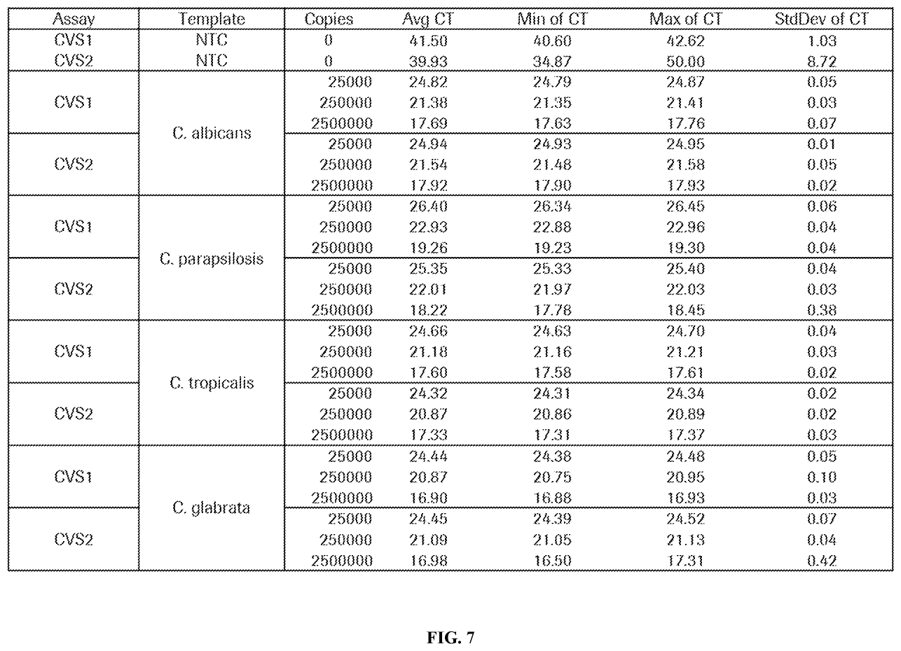

The rRNA gene was also used as the target gene in the CVS_rRNA_1 and CVS_rRNA_2 assays that target C. albicans, C. parapsilosis, C. tropicalis, and Candida glabrata. The CVS_rRNA_1 assay uses the forward primer of SEQ ID NO: 48, the reverse primer of SEQ ID NO: 49 and the probe of SEQ ID NO: 50. The CVS_rRNA_2 assay uses the forward primer of SEQ ID NO: 51, the reverse primer of SEQ ID NO: 52 and the probe of SEQ ID NO: 53. Both assays were tested with genomic DNA from C. albicans, C. parapsilosis, C. tropicalis, and C. glabrata at concentrations of 2.5e6, 2.5e5 and 2.5e4 copies per PCR reaction, and the results, expressed as Ct values, are shown in FIG. 7.

### Example 10 Multiplex PCR Assay

A multiplex PCR single well assay that simultaneously detects three BV-related bacteria, Lactobacillus spp., Gardnerella vaginalis, Atopobium vaginae, and Candida spp, including Candida krusei and Candida glabrata was performed using four different detection channels. The first channel detects the 16s rRNA of Gardnerella vaginalis. The second channel detects the D-LDH gene of Lactobacillus spp. The third channel detects tufA gene of Atopobium vaginae. The fourth channel detects a plurality of Candida including the 25s rRNA of Candida spp., and the rRNA ITS segments of Candida krusei and Candida glabrata. Select combination set of primers and probes used in the multiplex assay are shown in TABLE XII.

!TABLE XII

| Organism/Channel No. | Target Gene | Oligo Name | SEQ ID NO: |
|---|---|---|---|
| Gardnerella vaginalis/1 | 16s rRNA | GVAG_F1 | 11 |
| | | GVAG_R1 | 12 |
| | | GVAG_16S_Pb_l | 13 |
| Lactobacillus spp/2 | D-LDH | LBS_LDH_F_1 | 44 |
| | | LBS_LDH_R_2 | 45 |
| | | LBS_LDH_Pb_1 | 46 |
| Atopobium vaginae/3 | tufA | AVAG_tufA_F_3 | 20 |
| | | AVAG_tufA_R_3 | 21 |
| | | AVAG_tufA_Pb_4 | 72 |
| Candida spp./4 | 25s rRNA | CAPT_rRNA_F_2_ | 76 |
| | | CAPT_rRNA_R_2 | 77 |
| | | CAPT_rRNA_Pb_2 | 78 |
| Candida krusei/4 | 18s rRNA | KRUS_rRNA_F_3 | 79 |
| | | KRUS_rRNA_R_3 | 80 |
| | | KRUS_rRNA_Pb_3 | 81 |
| Candida glabrata/4 | rRNA ITS | GLAB_rRNA_F_1 | 63 |
| | | GLAB_rRNA_R_1 | 64 |
| | | GLAB_rRNA_Pb_1 | 65 |

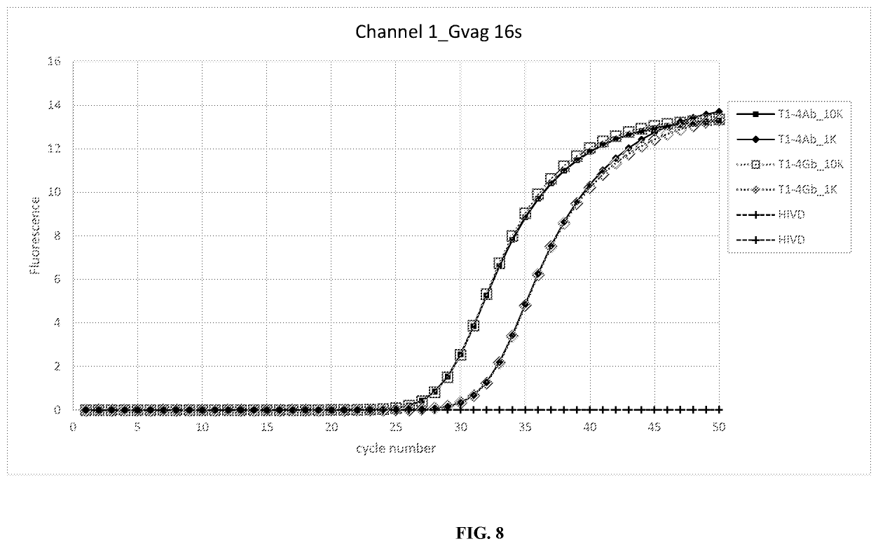

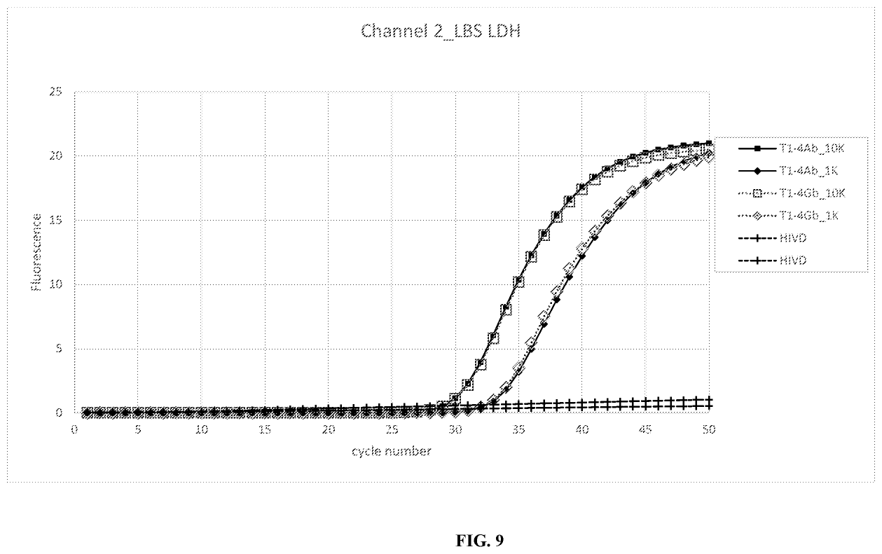

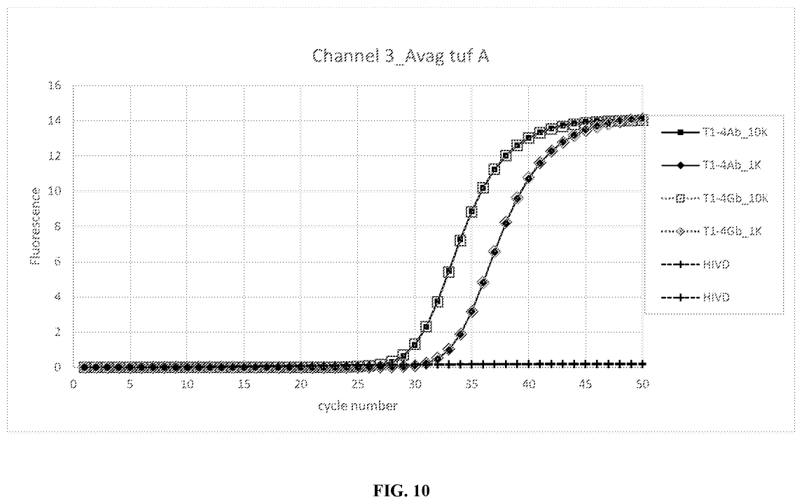

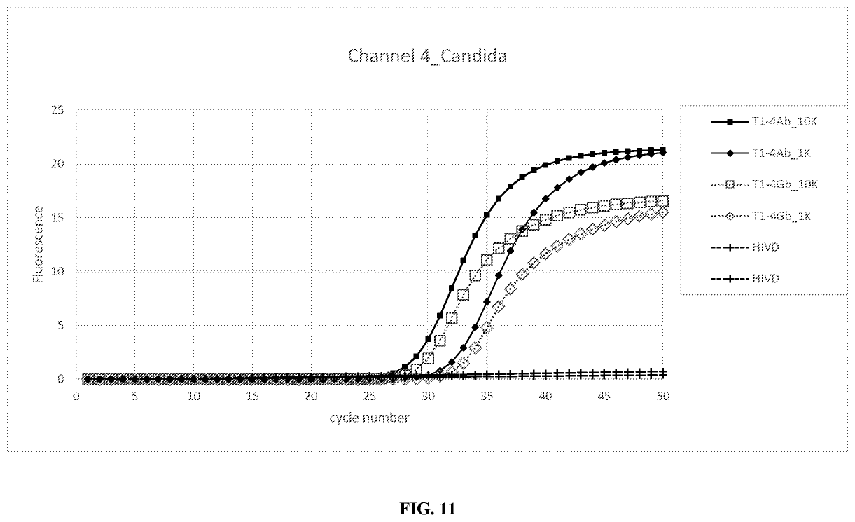

PCR assay reagents and conditions were used as described in Example 1 and the results of the experiment tested against various template samples at 10,000 (10K) and 1,000 (1K) copies are shown as growth curves in FIGS. 8, 9, 10 and 11 (for Candida spp. only) for Channels 1, 2, 3 and 4, respectively.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A method of detecting a plurality of Bacterial Vaginosis-related (BV-related) bacteria in a sample, wherein the plurality of BV-related bacteria are Lactobacillus spp., Gardnerella vaginalis, and at least one of the group selected from Atopobium vaginae, Megasphaera Type 1, Eggerthella spp., Prevotella spp., and Bacterial Vaginosis Associated Bacterium BVAB-2, the method comprising:
a) performing an amplifying step comprising contacting the sample with a set of primers to produce an amplification product if a nucleic acid from the BV-related bacteria is present in the sample;
b) performing a hybridizing step comprising contacting each amplification product with one or more detectable probes; and
c) detecting the presence of each amplification product, wherein the presence of the amplification product is indicative of the presence of the BV-related bacteria in the sample;
wherein the set of primers to produce an amplification product from Lactobacillus spp. comprises a forward primer selected from SEQ ID NOs: 38, 41, or 44, and a reverse primer selected from SEQ ID NOs: 39, 42 or 45, 47 or 73, and the one or more detectable probes is selected from SEQ ID NOs: 40, 43, 46 and 74;
wherein the set of primers to produce an amplification product from Gardnerella vaginalis comprises a forward primer selected from SEQ ID NOs: 1, 4, 5, 8 or 11, and a reverse primer selected from SEQ ID NOs: 2, 6, 9, or 12, and the one or more detectable probes is selected from SEQ ID NOs: 3, 7, 10 and 13; wherein the set of primers to produce an amplification product from Atopobium vaginae comprises a forward primer selected from SEQ ID NOs: 14, 17, 20, 67 or 70, and a reverse primer selected from SEQ ID NOs: 15, 18, 21, 68 or 71, and the one or more detectable probes is selected from SEQ ID NOs: 16, 19, 22, 66, 69 and 72;
wherein the set of primers to produce an amplification product from Megasphaera Type 1 comprises a forward primer selected from SEQ ID NOs: 23, 26 or 27, and a reverse primer selected from SEQ ID NOs: 24 or 28, and the one or more detectable probe is selected from SEQ ID NOs: 25 and 29;
wherein the set of primers to produce an amplification product from Eggerthella spp. comprises a forward primer selected from SEQ ID NOs: 84 or 87, and a reverse primer selected from SEQ ID NOs: 85 or 88, and the one or more detectable probe is selected from SEQ ID NOs: 86 and 89;
wherein the set of primers to produce an amplification product from Prevotella spp. comprises a forward primer selected from SEQ ID NOs: 90, 95, 96 or 97, and a reverse primer selected from SEQ ID NOs: 91, 92, 93, 98, 99 or 100, and the one or more detectable probe is selected from SEQ ID NOs: 94, 101 and 102; and
wherein the set of primers to produce an amplification product from BVAB-2 comprises a forward primer selected from SEQ ID NOs: 30 or 33, and a reverse primer selected from SEQ ID NOs: 31, 34 or 36, and the one or more detectable probe is selected from SEQ ID NOs: 32, 35 and 37.

2. The method of claim 1, wherein the plurality of BV-related bacteria are Lactobacillus spp., Gardnerella vaginalis, and Atopobium vaginae;
wherein the set of primers to produce an amplification product from Lactobacillus spp. comprises a forward primer selected from SEQ ID NOs: 38, 41, or 44, and a reverse primer selected from SEQ ID NOs: 39, 42 or 45, 47 or 73, and the one or more detectable probes is selected from SEQ ID NOs: 40, 43, 46 and 74;
wherein the set of primers to produce an amplification product from Gardnerella vaginalis comprises a forward primer selected from SEQ ID NOs: 1, 4, 5, 8 or 11, and a reverse primer selected from SEQ ID NOs: 2, 6, 9, or 12, and the one or more detectable probes is selected from SEQ ID NOs: 3, 7, 10 and 13; and
wherein the set of primers to produce an amplification product from Atopobium vaginae comprises a forward primer selected from SEQ ID NOs: 14, 17, 20, 67 or 70, and a reverse primer selected from SEQ ID NOs: 15, 18, 21, 68 or 71, and the one or more detectable probes is selected from SEQ ID NOs: 16, 19, 22, 66, 69 and 72.

3. The method of claim 2, wherein the set of primers to produce an amplification product from Lactobacillus spp. comprises a forward primer of SEQ ID NO: 44, and a reverse primer of SEQ ID NO: 45 and the one or more detectable probes is SEQ ID NO: 46; and the set of primers to produce an amplification product from Gardnerella vaginalis comprises a forward primer of SEQ ID NO: 11, and a reverse primer of SEQ ID NO: 12, and the one or more detectable probes is SEQ ID NO: 13; and the set of primers to produce an amplification product from Atopobium vaginae comprises a forward primer of SEQ ID NO: 20, and a reverse primer of SEQ ID NO: 21, and the one or more detectable probes is SEQ ID NO: 72.

4. The method of claim 1, wherein the hybridizing step comprises contacting the amplification product with the detectable probe that is labeled with a donor fluorescent moiety and a corresponding acceptor moiety; and the detecting step comprises detecting the presence or absence of fluorescence resonance energy transfer (FRET) between the donor fluorescent moiety and the acceptor moiety of the probe, wherein the presence or absence of fluorescence is indicative of the presence or absence of the BV-related bacteria in the sample.

5. The method of claim 4, wherein the amplification step employs a polymerase enzyme having 5′ to 3′ nuclease activity.

6. A method of detecting vulvovaginal candidiasis (VVC)-associated Candida species in a sample, wherein the VVC-associated Candida species comprises Candida albicans, Candida tropicalis Candida dubliniensis and Candida parapsilosis, (collectively referred as Candida spp.), and Candida krusei and Candida glabrata, the method comprising:
a) performing an amplifying step comprising contacting the sample with a set of primers to produce an amplification product if a nucleic acid from the VVC-associated Candida is present in the sample;
b) performing a hybridizing step comprising contacting each amplification product with one or more detectable probes; and
c) detecting the presence of each amplification product, wherein the presence of the amplification product is indicative of the presence of the VVC-associated Candida in the sample;
wherein the set of primers to produce an amplification product from Candida spp. comprises a forward primer selected from SEQ ID NOs: 48, 51, 54, 57 or 76, and a reverse primer selected from SEQ ID NOs: 49, 52, 55, 58 or 77, and the one or more detectable probes is selected from SEQ ID NOs: 50, 53, 56, 59, 75 and 78;
wherein the set of primers to produce an amplification product from Candida krusei comprises a forward primer selected from SEQ ID NOs: 60, 79 or 82, and a reverse primer selected from SEQ ID NOs: 61, 80 or 83, and the one or more detectable probes is selected from SEQ ID NOs: 62 and 81; and
wherein the set of primers to produce an amplification product from Candida glabrata comprises a forward primer of SEQ ID NO: 63, and a reverse primer of SEQ ID NO: 64, and the one or more detectable probes is SEQ ID NO; 65.

7. The method of claim 6, wherein the set of primers to produce an amplification product from Candida spp. comprises a forward primer of SEQ ID NO: 76, and a reverse primer of SEQ ID NO: 77 and the one or more detectable probes is SEQ ID NO: 78; and the set of primers to produce an amplification product from Candida krusei comprises a forward primer of SEQ ID NO: 79, and a reverse primer of SEQ ID NO: 80, and the one or more detectable probes is SEQ ID NO: 81.

8. The method of claim 6, wherein the hybridizing step comprises contacting the amplification product with the detectable probe that is labeled with a donor fluorescent moiety and a corresponding acceptor moiety; and the detecting step comprises detecting the presence or absence of fluorescence resonance energy transfer (FRET) between the donor fluorescent moiety and the acceptor moiety of the probe, wherein the presence or absence of fluorescence is indicative of the presence or absence of the VVC-associated Candida in the sample.

9. The method of claim 8, wherein the amplification step employs a polymerase enzyme having 5′ to 3′ nuclease activity.

10. A method of simultaneously detecting Lactobacillus spp., Gardnerella vaginalis, Atopobium vaginae, and at least one of the group selected from Candida spp., Candida krusei and Candida glabrata in a sample, the method comprising:
a) performing an amplifying step comprising contacting the sample with a set of primers to produce an amplification product if a nucleic acid from Lactobacillus spp., Gardnerella vaginalis, Atopobium vaginae, and at least one of the group selected from Candida spp., Candida krusei and Candida glabrata is present in the sample;
b) performing a hybridizing step comprising contacting each amplification product with one or more detectable probes; and
c) detecting the presence of each amplification product, wherein the presence of the amplification product is indicative of the presence of Lactobacillus spp., Gardnerella vaginalis, Atopobium vaginae, and at least one of the group selected from Candida spp., Candida krusei and Candida glabrata in the sample;
wherein the set of primers to produce an amplification product from Lactobacillus spp. comprises a forward primer of SEQ ID NO: 44, and a reverse primer of SEQ ID NO: 45 and the one or more detectable probes is SEQ ID NO: 46; and the set of primers to produce an amplification product from Gardnerella vaginalis comprises a forward primer of SEQ ID NO: 11, and a reverse primer of SEQ ID NO: 12, and the one or more detectable probes is SEQ ID NO: 13; and the set of primers to produce an amplification product from Atopobium vaginae comprises a forward primer of SEQ ID NO: 20, and a reverse primer of SEQ ID NO: 21, and the one or more detectable probes is SEQ ID NO: 72; and
wherein the set of primers to produce an amplification product from Candida spp. comprises a forward primer of SEQ ID NO: 76, and a reverse primer of SEQ ID NO: 77 and the one or more detectable probes is SEQ ID NO: 78; and the set of primers to produce an amplification product from Candida krusei comprises a forward primer of SEQ ID NO: 79, and a reverse primer of SEQ ID NO: 80, and the one or more detectable probes is SEQ ID NO: 81; and the set of primers to produce an amplification product from Candida glabrata comprises a forward primer of SEQ ID NO: 63, and a reverse primer of SEQ ID NO: 64, and the one or more detectable probes is SEQ ID NO; 65.

11. A kit for detecting Bacterial Vaginosis-related (BV-related) bacteria in a sample comprising:
a forward primer comprising a first oligonucleotide sequence selected from the group consisting of SEQ ID NOs: 1, 4, 5, 8, 11, 14, 17, 20, 23, 26, 27, 30, 33, 38, 41, 44, 67, 70, 84, 87, 90, 95, 96, and 97;
a reverse primer comprising a second oligonucleotide sequence selected from the group consisting of SEQ ID NOs: 2, 6, 9, 12, 15, 18, 21, 24, 28, 31, 34, 36, 39, 42, 45, 47, 68, 71, 73, 85, 88, 91, 92, 93, 98, 99, and 100; and
a third detectably labeled oligonucleotide sequence selected from the group consisting of SEQ ID NOs: 3, 7, 10, 13, 16, 66, 69, 19, 22, 72, 25, 29, 32, 35, 37, 40, 43, 46, 74, 86, 89, 94, 101, and 102, or a complement thereof, the third detectably labeled oligonucleotide sequence configured to hybridize to an amplicon generated by the forward primer and the reverse primer.

12. The kit of claim 11, wherein the third detectably labeled oligonucleotide sequence comprises a donor fluorescent moiety and a corresponding acceptor moiety.

13. The kit of claim 11, further comprising nucleoside triphosphates, nucleic acid polymerase, and buffers necessary for the function of the nucleic acid polymerase.

14. The kit of claim 11, wherein at least one of the first, second, and third oligonucleotide sequences comprises at least one modified nucleotide.

15. A kit for detecting vulvovaginal candidiasis (VVC)-associated Candida species in a sample, comprising:
a forward primer comprising a first oligonucleotide sequence selected from the group consisting of SEQ ID NOs: 48, 51, 54, 57, 76, 60, 79, 82, and 63;
a reverse primer comprising a second oligonucleotide sequence selected from the group consisting of SEQ ID NOs: 49, 52, 55, 58, 77, 61, 80, 83, and 64; and
a third detectably labeled oligonucleotide sequence selected from the group consisting of SEQ ID NOs: 50, 53, 75, 56, 59, 78, 62, 81, and 65, or a complement thereof, the third detectably labeled oligonucleotide sequence configured to hybridize to an amplicon generated by the forward primer and the reverse primer.

16. The kit of claim 15, wherein the third detectably labeled oligonucleotide sequence comprises a donor fluorescent moiety and a corresponding acceptor moiety.

17. The kit of claim 15, further comprising nucleoside triphosphates, nucleic acid polymerase, and buffers necessary for the function of the nucleic acid polymerase.

18. The kit of claim 15, wherein at least one of the first, second, and third oligonucleotide sequences comprises at least one modified nucleotide.


SEQUENCE LISTING (102 sequences)

SEQ ID NO: 1 - DNA, 20 bp, Artificial Sequence
  Forward Primer GVAG_tuf_F_1
  modified_base (20)..(20): t-butylbenzyl dA
agctcccaatcaacacccca

SEQ ID NO: 2 - DNA, 25 bp, Artificial Sequence
  Reverse Primer GVAG_tuf_R_1
  modified_base (25)..(25): t-butylbenzyl dC
cacggagaagaagaccagtgttatc

SEQ ID NO: 3 - DNA, 33 bp, Artificial Sequence
  Probe GVAG_tuf_Pb_1
  misc_feature: 5` Coumarin
  misc_feature: 3` Spacer-C3 Blocker
  misc_feature (10)..(11): BHQ-2 Quencher
agaccaccaccgtcacctctatcgagaccttcc

SEQ ID NO: 4 - DNA, 23 bp, Artificial Sequence
  Forward Primer GVAG_tuf_F_2
  modified_base (23)..(23): t-butylbenzyl dA
cccaatcaacaccccagttgaga

SEQ ID NO: 5 - DNA, 23 bp, Artificial Sequence
  Forward Primer GVAG_23s_F_4
  modified_base (23)..(23): t-butylbenzyl dC
ctgtaaggctgattccggtgatc

SEQ ID NO: 6 - DNA, 21 bp, Artificial Sequence
  Reverse Primer GVAG_23s_R_4
  modified_base (21)..(21): t-butylbenzyl dC
cgagttccttggccaggattc

SEQ ID NO: 7 - DNA, 29 bp, Artificial Sequence
  Probe GVAG_23s_Pb_4
  misc_feature: 5` Coumarin
  misc_feature: 3` Spacer-C3 Blocker
  misc_feature (10)..(11): BHQ-2 Quencher
caacgtgaggtgtgattccgctcgtaccc

SEQ ID NO: 8 - DNA, 19 bp, Artificial Sequence
  Forward Primer GVAG_23s_F_6
  modified_base (19)..(19): t-butylbenzyl dC
agtgacgcgggtaggtagc

SEQ ID NO: 9 - DNA, 22 bp, Artificial Sequence
  Reverse Primer GVAG_23s_R_5
  modified_base (22)..(22): t-butylbenzyl dA
gcaagcaggatcaccggaatca

SEQ ID NO: 10 - DNA, 28 bp, Artificial Sequence
  Probe GVAG_23s_Pb_5
  misc_feature: 5` Coumarin
  misc_feature: 3` Spacer-C3 Blocker
  misc_feature (10)..(11): BHQ-2 Quencher
ctgaggcgtgatgggtaggctgtaaggc

SEQ ID NO: 11 - DNA, 21 bp, Artificial Sequence
  Forward Primer GVAG_F1
  modified_base (21)..(21): t-butylbenzyl dA
tgctccagaatagctcttgga

SEQ ID NO: 12 - DNA, 22 bp, Artificial Sequence
  Reverse Primer GVAG_R1
  modified_base (22)..(22): t-butylbenzyl dA
gccactaaacactttcccaaca

SEQ ID NO: 13 - DNA, 26 bp, Artificial Sequence
  Probe GVAG_16s_Pb_1
  misc_feature: 5` Coumarin
  misc_feature: 3` Spacer-C3 Blocker
  misc_feature (10)..(11): BHQ-2 Quencher
tgctggatgctccaacttgacgcatg

SEQ ID NO: 14 - DNA, 20 bp, Artificial Sequence
  Forward Primer AVAG_23s_F_1
  modified_base (20)..(20): t-butylbenzyl dA
gctgagtccggttgggataa

SEQ ID NO: 15 - DNA, 23 bp, Artificial Sequence
  Reverse Primer AVAG_23s_R_1
  modified_base (12)..(12): 7-deaza dG
  modified_base (23)..(23): t-butylbenzyl dA
ttgtctacaaggggtcttaccaa

SEQ ID NO: 16 - DNA, 30 bp, Artificial Sequence
  Probe AVAG_23s_Pb_1
  misc_feature: 5` HEX
  misc_feature: 3` Spacer-C3 Blocker
  misc_feature (6)..(7): BHQ-2 Quencher
cgggaagcccatcccaagatgagttttcct

SEQ ID NO: 17 - DNA, 17 bp, Artificial Sequence
  Forward Primer AVAG_tufA_F_2
  modified_base (17)..(17): t-butylbenzyl dA
gcctggcgaccacgtaa

SEQ ID NO: 18 - DNA, 20 bp, Artificial Sequence
  Reverse Primer AVAG_tufA_R_2
  modified_base (20)..(20): t-butylbenzyl dA
tgtggccaccttcacgaata

SEQ ID NO: 19 - DNA, 37 bp, Artificial Sequence
  Probe AVAG_tufA_Pb_2
  misc_feature: 5` HEX
  misc_feature: 3` Spacer-C3 Blocker
  misc_feature (8)..(9): BHQ-2 Quencher
attcaccccattgctatggaggaaggtcttaagttcg

SEQ ID NO: 20 - DNA, 22 bp, Artificial Sequence
  Forward Primer AVAG_tufA_F_3
  modified_base (22)..(22): t-butylbenzyl dA
cactaaggttctctccgagcaa

SEQ ID NO: 21 - DNA, 25 bp, Artificial Sequence
  Reverse Primer AVAG_tufA_R_3
  modified_base (25)..(25): t-butylbenzyl dA
tcaatgtgagcaacgttaatggtaa

SEQ ID NO: 22 - DNA, 27 bp, Artificial Sequence
  Probe AVAG_tufA_Pb_3
  misc_feature: 5` HEX
  misc_feature: 3` Spacer-C3 Blocker
  misc_feature (6)..(7): BHQ-2 Quencher
cgacaaggctcctgaagagcgtcaacg

SEQ ID NO: 23 - DNA, 20 bp, Artificial Sequence
  Forward Primer MEGA1_16s_F_2
  modified_base (20)..(20): t-butylbenzyl dC
ccgcgtgagtgaagaaggtc

SEQ ID NO: 24 - DNA, 21 bp, Artificial Sequence
  Reverse Primer MEGA1_16s_R_2
  modified_base (21)..(21): t-butylbenzyl dC
ggtaccgtcacggacggatac

SEQ ID NO: 25 - DNA, 35 bp, Artificial Sequence
  MEGA1_16s_Pb_2
  misc_feature: 5` HEX
  misc_feature: 3` Spacer-C3 Blocker
  misc_feature (6)..(7): BHQ-2 Quencher
agctctgttatacgggacgaaaaagacggatgcca

SEQ ID NO: 26 - DNA, 21 bp, Artificial Sequence
  Forward Primer MEGA1_16s_F_4
  modified_base (21)..(21): t-butylbenzyl dA
ccgcaatggacgaaagtctga

SEQ ID NO: 27 - DNA, 22 bp, Artificial Sequence
  Forward Primer MEGA1_16s_F_6
  modified_base (22)..(22): t-butylbenzyl dC
tcttccgcaatggacgaaagtc

SEQ ID NO: 28 - DNA, 25 bp, Artificial Sequence
  Reverse Primer MEGA1_16s_R_6
  modified_base (25)..(25): t-butylbenzyl dA
gcatccgtctttttcgtcccgtata

SEQ ID NO: 29 - DNA, 30 bp, Artificial Sequence
  Probe MEGA1_16s_Pb_6
  misc_feature: 5` HEX
  misc_feature: 3` Spacer-C3 BNlocker
  misc_feature (9)..(10): BHQ-2 Quencher
caacgccgcgtgagtgaagaaggtcttcgg

SEQ ID NO: 30 - DNA, 20 bp, Artificial Sequence
  Forward Primer BVAB2_16s_F_1
  modified_base (20)..(20): t-butylbenzyl dA
cccgcacaagcagtggatta

SEQ ID NO: 31 - DNA, 23 bp, Artificial Sequence
  Reverse Primer BVAB2_16s_R_1
  modified_base (23)..(23): t-butylbenzyl dA
tgtctctctgtctccgaagagaa

SEQ ID NO: 32 - DNA, 34 bp, Artificial Sequence
  Probe BVAB2_16s_Pb_1
  misc_feature: 5` HEX
  misc_feature: 3` Spacer-C3 Blocker
  misc_feature (11)..(12): BHQ-2 Quencher
accttaccaggacttgacatcctctgacgattca

SEQ ID NO: 33 - DNA, 27 bp, Artificial Sequence
  Forward Primer BVAB2_16s_F_3
  modified_base (27)..(27): t-butylbenzyl dA
agtgtgatgtttaaatccaaggcttaa

SEQ ID NO: 34 - DNA, 28 bp, Artificial Sequence
  Reverse Primer BVAB2_16s_R_3
  modified_base (28)..(28): t-butylbenzyl dA
gtgttcctcctaatatctacgcatttta

SEQ ID NO: 35 - DNA, 36 bp, Artificial Sequence
  Probe BVAB2_16s_Pb_3
  misc_feature: 5` HEX
  misc_feature: 3` Spacer-C3 Blocker
  misc_feature (11)..(12): BHQ-2 Quencher
caaactgtttagcttgagtgctggagaggatagtgg

SEQ ID NO: 36 - DNA, 28 bp, Artificial Sequence
  Reverse Primer BVAB2_16s_R_4
  modified_base (28)..(28): t-butylbenzyl dC
gttcctcctaatatctacgcattttacc

SEQ ID NO: 37 - DNA, 40 bp, Artificial Sequence
  Probe BVAB2_16s_Pb_4
  misc_feature: 5` HEX
  misc_feature: 3` Spacer-C3 Blocker
  misc_feature (7)..(8): BHQ-2 Quencher
tcattacaaactgtttagcttgagtgctggagaggatagt

SEQ ID NO: 38 - DNA, 25 bp, Artificial Sequence
  Forward Primer LBS_23s_F_1
  modified_base (25)..(25): t-butylbenzyl dA
tcaggggttactgaatccagataaa

SEQ ID NO: 39 - DNA, 25 bp, Artificial Sequence
  Reverse Primer LBS_23s_R_1
  modified_base (25)..(25): t-butylbenzyl dA
acttagcatagatttggggacctta

SEQ ID NO: 40 - DNA, 35 bp, Artificial Sequence
  Probe LBS_23s_Pb_1
  misc_feature: 5` FAM
  misc_feature: 3` Spacer-C3 Blocker
  misc_feature (9)..(10): BHQ-2 Quencher
agtcagactgcgagtgataagatccgtagtcgaaa

SEQ ID NO: 41 - DNA, 21 bp, Artificial Sequence
  Forward Primer LBS_23s_F_2
  modified_base (21)..(21): t-butylbenzyl dA
gtgagaatcctatccgccgaa

SEQ ID NO: 42 - DNA, 21 bp, Artificial Sequence
  Reverse Primer LBS_23s_R_2
  modified_base (21)..(21): t-butylbenzyl dA
cttgccttaggtcccgactta

SEQ ID NO: 43 - DNA, 22 bp, Artificial Sequence
  Probe LBS_23s_Pb_2
  misc_feature: 5` FAM
  misc_feature: 3` Spacer-C3 Blocker
  misc_feature (7)..(8): BHQ-2 Quencher
ttcctggggcaggctcgtccgc

SEQ ID NO: 44 - DNA, 23 bp, Artificial Sequence
  Forward Primer LBS_LDH_F_1
  modified_base (23)..(23): t-butylbenzyl dA
gctcgtgtattacgtcaagacaa

SEQ ID NO: 45 - DNA, 27 bp, Artificial Sequence
  Reverse Primer LBS_LDH_R_2
  modified_base (27)..(27): t-butylbenzyl dC
gaagatatcgtaagcaataacctttgc

SEQ ID NO: 46 - DNA, 36 bp, Artificial Sequence
  Probe LBS_LDH_Pb_1
  misc_feature: 5` FAM
  misc_feature: 3` Spacer-C3 Blocker
  misc_feature (6)..(7): BHQ-2 Quencher
tcgtgaccaagttgttggtgttgtaggtactggtca

SEQ ID NO: 47 - DNA, 20 bp, Artificial Sequence
  Reverse Primer LBS_LDH_R_1
  modified_base (20)..(20): t-butylbenzyl dC
cctttgcgccgaaaccttcc

SEQ ID NO: 48 - DNA, 25 bp, Artificial Sequence
  Forward Primer CVS_rRNA_F_1
  modified_base (25)..(25): t-butylbenzyl dC
caacggatctcttggttctcgcatc

SEQ ID NO: 49 - DNA, 19 bp, Artificial Sequence
  Reverse Primer CVS_rRNA_R_1
  modified_base (19)..(19): t-butylbenzyl dA
gggcgcaatgtgcgttcaa

SEQ ID NO: 50 - DNA, 36 bp, Artificial Sequence
  Probe CVS_rRNA_Pb_1
  misc_feature: 5` JA270
  misc_feature: 3` Spacer-C3 Blocker
  misc_feature (10)..(11): BHQ-2 Quencher
ctgcaattcatattacgtatcgcatttcgctgcgtt

SEQ ID NO: 51 - DNA, 21 bp, Artificial Sequence
  Forward Primer CVS_rRNA_F_2
  modified_base (21)..(21): t-butylbenzyl dA
gcgagagaccgatagcgaaca

SEQ ID NO: 52 - DNA, 24 bp, Artificial Sequence
  Reverse Primer CVS_rRNA_R_2
  modified_base (24)..(24): t-butylbenzyl dC
gcccttccctttcaacaatttcac

SEQ ID NO: 53 - DNA, 45 bp, Artificial Sequence
  Probe CVS_rRNA_Pb_2
  misc_feature: 5` JA270
  misc_feature: 3` Spacer-C3 Blocker
  misc_feature (10)..(11): BHQ-2 Quencher
tttcactctcttttcaaagttcttttcatctttccatcactgtac

SEQ ID NO: 54 - DNA, 32 bp, Artificial Sequence
  Forward Primer CAPT_rRNA_F_1
  modified_base (32)..(32): t-butylbenzyl dC
aatgcgatacgtaatatgaattgcagatattc

SEQ ID NO: 55 - DNA, 24 bp, Artificial Sequence
  Reverse Primer CAPT_rRNA_R_1
  modified_base (24)..(24): t-butylbenzyl dA
tttgagggagaaatgacgctcaaa

SEQ ID NO: 56 - DNA, 33 bp, Artificial Sequence
  Probe CAPT_rRNA_Pb_1
  misc_feature: 5` JA270
  misc_feature: 3` Spacer-C3 Blocker
  misc_feature (10)..(11): BHQ-2 Quencher
atcgaatctttgaacgcacattgcgccctttgg

SEQ ID NO: 57 - DNA, 27 bp, Artificial Sequence
  Forward Primer CAPT_rRNA_F_6
  modified_base (27)..(27): t-butylbenzyl dA
aagaaaccaacagggattgccttagta

SEQ ID NO: 58 - DNA, 24 bp, Artificial Sequence
  Reverse Primer CAPT_rRNA_R_6
  modified_base (24)..(24): t-butylbenzyl dA
tcatctcatcgcacgggattctca

SEQ ID NO: 59 - DNA, 44 bp, Artificial Sequence
  Probe CAPT_rRNA_Pb_6
  misc_feature: 5` JA270
  misc_feature: 3` Spacer-C3 Blocker
  misc_feature (10)..(11): BHQ-2 Quencher
agttgtaatttgaagaaggtatctttgggtctggctcttgtcta

SEQ ID NO: 60 - DNA, 23 bp, Artificial Sequence
  Forward Primer KRUS_rRNA_F_1
  modified_base (23)..(23): t-butylbenzyl dA
tcgtgaatcatcgagttcttgaa

SEQ ID NO: 61 - DNA, 16 bp, Artificial Sequence
  Reverse Primer KRUS_rRNA_R_1
  modified_base (16)..(16): t-butylbenzyl dC
acacgtcgtccgctcc

SEQ ID NO: 62 - DNA, 26 bp, Artificial Sequence
  Probe KRUS_rRNA_Pb_1
  misc_feature: 5` JA270
  misc_feature: 3` Spacer-c3 Blocker
  misc_feature (10)..(11): BHQ-2 Quencher
tttgagcgtcgtttccatcttgcgcg

SEQ ID NO: 63 - DNA, 25 bp, Artificial Sequence
  Forward Primer GLAB_rRNA_F_1
  modified_base (25)..(25): t-butylbenzyl dA
aagtaaactccactgtgtgtagtaa

SEQ ID NO: 64 - DNA, 20 bp, Artificial Sequence
  Reverse Primer GLAB_rRNA_R_1
  modified_base (20)..(20): t-butylbenzyl dA
agtgtgcgtggatctctcta

SEQ ID NO: 65 - DNA, 34 bp, Artificial Sequence
  Probe GLAB_rRNA_Pb_1
  misc_feature: 5` JA270
  misc_feature: 3` Spacer-C3 Blocker
  misc_feature (10)..(11): BHQ-2 Quencher
agtgtcgagtcgtgtgataaaacacctcctttgg

SEQ ID NO: 66 - DNA, 27 bp, Artificial Sequence
  Probe AVAG_23s_Pb_2_H_Q6
  misc_feature: 5` HEX
  misc_feature: 3` Spacer-C3 Blocker
  misc_feature (6)..(7): BHQ-2 Quencher
aagcatctaagcgggaagcccatccca

SEQ ID NO: 67 - DNA, 19 bp, Artificial Sequence
  Forward Primer AVAG_23S_F_5
  modified_base (19)..(19): t-butylbenzyl dA
gccccagtcaaactaccca

SEQ ID NO: 68 - DNA, 21 bp, Artificial Sequence
  Reverse Primer AVAG_23S_R_5
  modified_base (3)..(3): 7-deaza dG
  modified_base (21)..(21): t-butylbenzyl dA
tgggagactgtgaactggaga

SEQ ID NO: 69 - DNA, 29 bp, Artificial Sequence
  Probe AVAG_23S_Pb_5
  misc_feature: 5` HEX
  misc_feature: 3` Spacer-C3 Blocker
  modified_base (4)..(4): 7-deaza dG
  misc_feature (7)..(8): BHQ-2 Quencher
  modified_base (27)..(27): 7-deaza dG
aagggttaggatgccgaaacgtcgagggt

SEQ ID NO: 70 - DNA, 19 bp, Artificial Sequence
  Forward Primer AVAG_23S_F_3
  modified_base (19)..(19): t-butylbenzyl dA
cggtccccttcccggatta

SEQ ID NO: 71 - DNA, 23 bp, Artificial Sequence
  Reverse Primer AVAG_23S_R_6
  modified_base (23)..(23): t-butylbenzyl dA
cgtgaacctttactgcagcttga

SEQ ID NO: 72 - DNA, 26 bp, Artificial Sequence
  Probe AVAG_tufA_Pb_4
  misc_feature: 5` HEX
  misc_feature: 3` Spacer-C3 Blocker
  misc_feature (7)..(8): BHQ-2 Quencher
aggctcctgaagagcgtcaacgtggt

SEQ ID NO: 73 - DNA, 28 bp, Artificial Sequence
  Reverse Primer LBS_LDH_R_3
  modified_base (28)..(28): t-butylbenzyl dA
cataaatacttgaccaatgtgaccagta

SEQ ID NO: 74 - DNA, 36 bp, Artificial Sequence
  Probe LBS_LDH_PB_3
  misc_feature: 5` FAM
  misc_feature: 3` Spacer-C3 Blocker
  misc_feature (8)..(9): BHQ-2 Quencher
tgaagttcgtgaccaagttgttggtgttgtaggtac

SEQ ID NO: 75 - DNA, 45 bp, Artificial Sequence
  Probe CVS_rRNA_Pb_3
  misc_feature: 5` JA270
  misc_feature: 3` Spacer-C3 Blocker
  misc_feature (9)..(10): BHQ-2 Quencher
tttcactctcttttcaaagtgcttttcatctttccttcacagtac

SEQ ID NO: 76 - DNA, 29 bp, Artificial Sequence
  Forward Primer CAPT_rRNA_F_2
  modified_base (29)..(29): t-butylbenzyl dC
agtccgagttgtaatttgaagaaggtatc

SEQ ID NO: 77 - DNA, 23 bp, Artificial Sequence
  Reverse Primer CAPT_rRNA_R_2
  modified_base (23)..(23): t-butylbenzyl dC
tgcattcccaaacaactcgactc

SEQ ID NO: 78 - DNA, 28 bp, Artificial Sequence
  Probe CAPT_rRNA_Pb_2
  misc_feature: 5` JA270
  misc_feature: 3` Spacer-C3 Blocker
  misc_feature (10)..(11): BHQ-2 Quencher
acgtcacagagggtgagaatcccgtgcg

SEQ ID NO: 79 - DNA, 24 bp, Artificial Sequence
  Forward Primer KRUS_rRNA_F_3
  modified_base (24)..(24): t-butylbenzyl dA
ggtaattccagctccaatagcgta

SEQ ID NO: 80 - DNA, 25 bp, Artificial Sequence
  Reverse Primer KRUS_rRNA_R_3
  modified_base (25)..(25): t-butylbenzyl dC
gcctgctttgaacactctaatttcc

SEQ ID NO: 81 - DNA, 27 bp, Artificial Sequence
  Probe KRUS_rRNA_PB_3
  misc_feature: 5` JA270
  misc_feature: 3` Spacer-C3 Blocker
  misc_feature (8)..(9): BHQ-2 Quencher
taagcactgttgcggccgggtctttcc

SEQ ID NO: 82 - DNA, 25 bp, Artificial Sequence
  Forward Primer KRUS_rRNA_F_4
  modified_base (25)..(25): t-butylbenzyl dA
agttgttgcagttaaaaagctcgta

SEQ ID NO: 83 - DNA, 33 bp, Artificial Sequence
  Reverse Primer KRUS_rRNA_R_4
  modified_base (33)..(33): t-butylbenzyl dC
gcgtcctattctattattccatgctaatatatc

SEQ ID NO: 84 - DNA, 19 bp, Artificial Sequence
  Forward Primer Egg_16s_F_1
  modified_base (6)..(6): 7-deaza dG
  modified_base (19)..(19): t-butylbenzyl dC
aactgggaggctcgagttc

SEQ ID NO: 85 - DNA, 23 bp, Artificial Sequence
  Reverse Primer Egg_16s_R_1
  modified_base (23)..(23): t-butylbenzyl dC
ccatcggtgttcttcccgatatc

SEQ ID NO: 86 - DNA, 25 bp, Artificial Sequence
  Probe Egg_16s_Pb_1
  misc_feature: 5` HEX
  misc_feature: 3` Spacer-C3 Blocker
  misc_feature (6)..(7): BHQ-2 Quencher
aggcaggcggaattcccggtgtagc

SEQ ID NO: 87 - DNA, 20 bp, Artificial Sequence
  Forward Primer Egg_16s_F_2
  modified_base (20)..(20): t-butylbenzyl dA
ggaggctcgagttcggtaga

SEQ ID NO: 88 - DNA, 21 bp, Artificial Sequence
  Reverse Primer Egg_16s_R_2
  modified_base (21)..(21): t-butylbenzyl dA
ccatcggtgttcttcccgata

SEQ ID NO: 89 - DNA, 23 bp, Artificial Sequence
  Probe Egg_16s_Pb_2
  misc_feature: 5` HEX
  misc_feature: 3` Spacer-C3 Blocker
  misc_feature (7)..(8): BHQ-2 Quencher
acaccgggaattccgcctgcctc

SEQ ID NO: 90 - DNA, 18 bp, Artificial Sequence
  Forward Primer Prev_16s_F_1
  modified_base (6)..(6): 7-deaza dG
  modified_base (18)..(18): t-butylbenzyl dA
ggatggggatgcgtctga

SEQ ID NO: 91 - DNA, 21 bp, Artificial Sequence
  Reverse Primer Prev_16s_R_1
  modified_base (21)..(21): t-butylbenzyl dC
gtttggaccgtgtctcagttc

SEQ ID NO: 92 - DNA, 20 bp, Artificial Sequence
  Reverse Primer Prev_16s_R_2
  modified_base (20)..(20): t-butylbenzyl dC
cctcccgtaggagtttggac

SEQ ID NO: 93 - DNA, 20 bp, Artificial Sequence
  Reverse Primer Prev_16s_R_3
  modified_base (20)..(20): t-butylbenzyl dA
gcctcccgtaggagtttgga

SEQ ID NO: 94 - DNA, 29 bp, Artificial Sequence
  Probe Prev_16s_Pb_2
  misc_feature: 5` HEX
  misc_feature: 3` Spacer-C3 Blocker
  misc_feature (7)..(8): BHQ-2 Quencher
tcagtaggggttctgagaggaaggtcccc

SEQ ID NO: 95 - DNA, 20 bp, Artificial Sequence
  Forward Primer Prev_16s_F_4
  modified_base (20)..(20): t-butylbenzyl dA
tcaaatcagcacggccctta

SEQ ID NO: 96 - DNA, 20 bp, Artificial Sequence
  Forward Primer Prev_16s_F_5
  modified_base (20)..(20): t-butylbenzyl dA
ccaccgtaaggtgtgaggaa

SEQ ID NO: 97 - DNA, 20 bp, Artificial Sequence
  Forward Primer Prev_16s_F_6
  modified_base (20)..(20): t-butylbenzyl dA
ccaccgcaaggtgcgaggaa

SEQ ID NO: 98 - DNA, 15 bp, Artificial Sequence
  Reverse Primer Prev_16s_R_4
  modified_base (15)..(15): t-butylbenzyl dC
gcacccccggctttc

SEQ ID NO: 99 - DNA, 16 bp, Artificial Sequence
  Reverse Primer Prev_16s_R_5
  modified_base (16)..(16): t-butylbenzyl dC
ggcacccccggctttc

SEQ ID NO: 100 - DNA, 19 bp, Artificial Sequence
  Reverse Primer Prev_16s_R_6
  modified_base (19)..(19): t-butylbenzyl dA
catggctgatgcgcgatta

SEQ ID NO: 101 - DNA, 31 bp, Artificial Sequence
  Probe Prev_16s_Pb_4
  misc_feature: 5` HEX
  misc_feature: 3` Spacer-C3 Blocker
  misc_feature (7)..(8): BHQ-2 Quencher
accccacgaagctggattcgctagtaatcgc

SEQ ID NO: 102 - DNA, 29 bp, Artificial Sequence
  Probe Prev_16s_Pb_5
  misc_feature: 5` HEX
  misc_feature: 3` Spacer-C3 Blocker
  modified_base (1)..(1): 7-deaza dG
  misc_feature (8)..(9): BHQ-2 Quencher
ggtctgcaacccgaccccacgaagctgga